(19) United States
(12) Patent Application Publication
(10) Pub. No.: US 2013/0030522 A1
(43) Pub. Date: Jan. 31, 2013

(54) DEVICES AND METHODS FOR HEART TREATMENTS

(71) Applicants: Stanton J. Rowe, Newport Beach, CA (US); Assaf Bash, Benyamina-Givat Ada (IL); John F. Migliazza, Belmont Shore, CA (US); Ricardo Villarreal, Mountain View, CA (US); Dan Rottenberg, Haifa (IL); Boaz Manash, Givat-Ada (IL)

(72) Inventors: Stanton J. Rowe, Newport Beach, CA (US); Assaf Bash, Benyamina-Givat Ada (IL); John F. Migliazza, Belmont Shore, CA (US); Ricardo Villarreal, Mountain View, CA (US); Dan Rottenberg, Haifa (IL); Boaz Manash, Givat-Ada (IL)

(21) Appl. No.: 13/162,391

(22) Filed: Jun. 16, 2011

Related U.S. Application Data
(60) Provisional application No. 61/355,437, filed on Jun. 16, 2010.

(51) Int. Cl. A61F 2/04; A61F 2/24

(52) U.S. Cl. 623/2.36; 600/16

(57) ABSTRACT

A method and device for treating a heart by assisting one or more heart chambers to expand during diastole. The method comprises providing a plurality of anchoring members; providing an elongate member and a release mechanism connected to the elongate member, the release mechanism being configured to releasably engage with each of the plurality of anchoring members; the elongate member being configured to store energy exerted by a heart chamber during systole, and release the stored energy during diastole to assist the heart chamber to return to an uncompressed state; selecting one of the plurality of anchoring members; positioning the elongate member transverse a chamber of the heart; and engaging the release mechanism with the selected anchoring member so as to releasably attach the elongate member to the selected anchoring member.

### CROSS REFERENCES TO THE RELATED APPLICATIONS

This application claims priority under 35 U.S.C. §119(e) (1) to the provisional patent Application No. 61/355,437 filed on Jun. 16, 2010, entitled “Devices and Methods for Heart Treatment”, the disclosure of which is incorporated herein by reference in its entirety.

### FIELD

The present disclosure relates to devices and associated methods for treating and improving the performance of a heart. More particularly, the present disclosure relates to devices and methods that passively assist to reshape a dysfunctional heart to improve its performance. For example, in some embodiments, the apparatus of the present disclosure may be directed toward reducing the wall stress in the failing heart. In other embodiments, the devices and methods disclosed herein may be used to treat a heart valve, such as, for example, a mitral valve.

### BACKGROUND

The syndrome of heart failure is a common course for the progression of many forms of heart disease. Heart failure may be considered to be the condition in which an abnormality of cardiac function is responsible for the inability of the heart to pump blood at a rate commensurate with the requirements of the metabolizing tissues, or can do so only at an abnormally elevated filling pressure. There are many specific disease processes that can lead to heart failure with a resulting difference in pathophysiology of the failing heart, such as the dilatation of the left ventricular chamber. Etiologies that can lead to this form of failure include idiopathic cardiomyopathy, viral cardiomyopathy, and ischemic cardiomyopathy.

The process of ventricular dilatation is generally the result of chronic volume overload or specific damage to the myocardium. In a normal heart that is exposed to long term increased cardiac output requirements, for example, that of an athlete, there is an adaptive process of slight ventricular dilation and muscle myocyte hypertrophy. In this way, the heart fully compensates for the increased cardiac output requirements. With damage to the myocardium or chronic volume overload, however, there are increased requirements put on the contracting myocardium to such a level that this compensated state is never achieved and the heart continues to dilate.

The basic problem with a large dilated left ventricle is that there is a significant increase in wall tension and/or stress both during diastolic filling and during systolic contraction. In a normal heart, the adaptation of muscle hypertrophy (thickening) and ventricular dilatation maintain a fairly constant wall tension for systolic contraction. However, in a failing heart, the ongoing dilatation is greater than the hypertrophy and the result is a rising wall tension requirement for systolic contraction. This is felt to be an ongoing insult to the muscle myocyte resulting in further muscle damage. The increase in wall stress is also true for diastolic filling. Additionally, because of the lack of cardiac output, there is generally a rise in ventricular filling pressure from several physiologic mechanisms. Moreover, in diastole there is both a diameter increase and a pressure increase over normal, both contributing to higher wall stress levels. The increase in diastolic wall stress is felt to be the primary contributor to ongoing dilatation of the chamber.

Therefore there is a need to devise effective techniques that could improve valve function without the need for cardiopulmonary bypass and without requiring major remodeling of the valve. In particular, passive techniques to change the shape of the heart chamber and/or associated valve and reduce regurgitation while maintaining substantially normal leaflet motion may be desirable. Further, advantages may be obtained by a technique that reduces the overall time a patient is in surgery and under the influence of anesthesia. It also may be desirable to provide a technique for treating valve insufficiency that reduces the risk of bleeding associated with anticoagulation requirements of cardiopulmonary bypass. In addition, a technique that can be employed on a beating heart would allow the practitioner an opportunity to assess the efficacy of the treatment and potentially address any inadequacies without the need for additional bypass support

### SUMMARY

The embodiments of the present disclosure have several features, no single one of which is solely responsible for their desirable attributes. Without limiting the scope of the present embodiments as expressed by the claims that follow, their more prominent features will now be discussed briefly. After considering this discussion, and particularly after reading the section entitled “Detailed Description”, one will understand how the features of the present embodiments provide advantages, which include a non-pharmacological, passive method and device for the treatment of a failing heart. The method and the heart treatment device is configured to reduce the tension in the walls of a heart. It is believed to reverse, stop or slow the disease process of a failing heart as it reduces the energy consumption of the failing heart, decrease in isovolumetric contraction, increases sarcomere shortening during contraction and an increase in isotonic shortening in turn increases stroke volume. The device reduces wall tension during diastole (preload) and systole.

In one embodiment, a method for improving the function of a heart is provided. The method comprises providing a plurality of anchoring members; providing an elongate member and a release mechanism connected to the elongate member, the release mechanism being configured to releasably engage with each of the plurality of anchoring members; the elongate member being configured to store energy exerted by a heart chamber during systole, and release the stored energy during diastole to assist the heart chamber to return to an uncompressed state; selecting one of the plurality of anchoring members; positioning the elongate member transverse a chamber of the heart; and engaging the release mechanism with the selected anchoring member so as to releasably attach the elongate member to the selected anchoring member.

In another embodiment, a heart treatment device is provided. The heart treatment device for improving the function of the heart comprises a plurality of anchoring members; an elongate member configured to be positioned transverse a chamber of the heart; where the elongate member has a substantially rigid distal end, a substantially rigid proximal end and a substantially elastic portion between the distal end and the proximal end; and a release mechanism connected to the elongate member, the release mechanism being configured to releasably engage with each of a plurality of anchoring members having differing configurations to releasably attach the elongate member to each of the plurality of anchoring members one at a time.

The features, functions, and advantages of the present embodiments can be achieved independently in various embodiments, or may be combined in yet other embodiments.

### BRIEF DESCRIPTION OF THE DRAWINGS

The embodiments of the present disclosure will now be discussed in detail with an emphasis on highlighting the advantageous features. These embodiments depict the novel and non-obvious device for improving the heart function shown in the accompanying drawings, which are for illustrative purposes only. These drawings include the following figures, in which like numerals indicate like parts.

FIG. 1A is a superior, short axis, cross-sectional view of a human heart during diastole, showing a mitral valve splint extending through the heart and aligned generally orthogonal to the arcuate opening of the mitral valve;

FIG. 1B is a lateral, long axis, cross-sectional view of the human heart and an exemplary embodiment of mitral valve splint of FIG. 1A;

FIG. 1C is an anterior, long axis view of the human heart and an exemplary embodiment of a mitral valve splint of FIG. 1A;

FIG. 2A is a superior, short axis, cross-sectional view of a human heart showing an incompetent mitral valve during systole;

FIG. 2B is a superior, short axis, cross-sectional view of the human heart of FIG. 2A showing the formerly incompetent mitral valve during systole corrected with an exemplary embodiment of a mitral valve splint;

FIGS. 3A-3C are side and perspective views of an exemplary embodiment of an anterior pad for use with the mitral valve splint shown in FIG. 1;

FIGS. 4A-4G are side and perspective views of an exemplary embodiment of a posterior pad for use with the mitral valve splint shown in FIG. 1;

FIGS. 4H-4P are schematic illustration of exemplary embodiments of the posterior pad, according to one embodiment;

FIG. 5A is a perspective view of an exemplary embodiment of a mitral valve splint delivery system including a positioning and alignment device (shown in the closed position) and a needle delivery assembly;

FIG. 5B is a perspective view of a portion of the delivery system of FIG. 5A, shown in the open position;

FIG. 5C is a schematic illustration of exemplary embodiments of the needle delivery assembly;

FIGS. 5D and 5E are perspective views of the anterior and posterior vacuum chambers, respectively, of the positioning and alignment device shown in FIG. 5A;

FIGS. 5F and 5G are exploded views of the anterior and posterior vacuum chambers, of FIGS. 5D and 5E, respectively;

FIG. 5H is a perspective view of an exemplary embodiment of a rotating insert for use in the posterior vacuum chamber of the mitral valve delivery system shown in FIG. 5A;

FIG. 5I is a perspective view of a capture plate for use in the posterior vacuum chamber of the mitral valve delivery system shown in FIG. 5A;

FIG. 5J is a schematic plan view of the delivery system of FIG. 5A with the positioning and alignment device disposed on the heart and the needle delivery assembly fully inserted through the heart;

FIGS. 6A-6D are schematic illustrations of an exemplary embodiment of a septal delivery system and method for a mitral valve splint;

FIGS. 7A-7E are schematic illustrations of an exemplary embodiment of an alternative septal delivery system and method for a mitral valve splint;

FIGS. 8A-8F are schematic illustrations of an exemplary embodiment of an endovascular septal delivery system and method for a mitral valve splint;

FIGS. 9A-9D are perspective views of an exemplary embodiment of an expandable pad and associated components for use with the mitral valve splints of FIGS. 6-8;

FIGS. 10A-10C are schematic views of an exemplary embodiment of an alternative expandable pad for use with the septal mitral valve splints of FIGS. 6-8;

FIG. 11 is a lateral, long axis, cross-sectional view of a human heart and an exemplary embodiment of a heart treatment device, in accordance with an aspect of the present disclosure;

FIG. 12 a lateral, long axis, cross-sectional view of a human heart and an exemplary embodiment of another heart treatment device, in accordance with another aspect of the present disclosure;

FIG. 13 is a lateral, long axis, cross-sectional view of a human heart and an exemplary embodiment of another heart treatment device, in accordance with a further aspect of the present disclosure; and

FIG. 14 is a lateral, long axis, cross-sectional view of a human heart and an exemplary embodiment of another heart treatment device, in accordance with a further aspect of the present disclosure.

Aside from the structural and procedural arrangements set forth above, the invention could include a number of other arrangements, such as those explained hereinafter. It is to be understood that both the foregoing and the following descriptions are exemplary. The accompanying drawings are included to provide a further understanding of the invention and are incorporated in and constitute a part of this specification. The drawings illustrate exemplary embodiments of the invention and, together with the description, serve to explain certain principles.

### DETAILED DESCRIPTION

The various aspects of the devices and methods described herein generally pertain to devices and methods for treating heart conditions, including, for example, dilatation, valve incompetencies, including mitral valve leakage, and other similar heart failure conditions. Each disclosed device may operate passively in that, once placed in the heart, it does not require an active stimulus, either mechanical, electrical, or otherwise, to function. Implanting one or more of the devices operates to assist in the apposition of heart valve leaflets to improve valve function.

In addition, these devices may either be placed in conjunction with other devices that, or may themselves function to, alter the shape or geometry of the heart, locally and/or globally, and thereby further increase the heart's efficiency. That is, the heart experiences an increased pumping efficiency through an alteration in its shape or geometry and concomitant reduction in stress on the heart walls, and through an improvement in valve function.

However, the devices disclosed herein for improving valve function can be “stand-alone” devices, that is, they do not necessarily have to be used in conjunction with additional devices for changing the shape of a heart chamber or otherwise reducing heart wall stress. It also is contemplated that a device for improving valve function may be placed relative to the heart without altering the shape of the chamber, and only altering the shape of the valve itself.

The devices and methods described herein offer numerous advantages over the existing treatments for various heart conditions, including valve incompetencies. The devices are relatively easy to manufacture and use, and the surgical techniques and tools for implanting the devices do not require the invasive procedures of current surgical techniques. For instance, the surgical technique does not require removing portions of the heart tissue, nor does it necessarily require opening the heart chamber or stopping the heart during operation. For these reasons, the surgical techniques for implanting the devices disclosed herein also are less risky to the patient than other techniques. The less invasive nature of these surgical techniques and tools may also allow for earlier intervention in patients with heart failure and/or valve incompetencies.

The devices and methods described herein involve geometric reshaping of the heart and treating valve incompetencies. In certain aspects of the devices and methods described herein, substantially the entire chamber geometry is altered to return the heart to a more normal state of stress. Models of this geometric reshaping, which includes a reduction in radius of curvature of the chamber walls with ventricular splints, may be found in U.S. Pat. Nos. 5,961,440 and 6,050,936, the entire disclosures of these patents are incorporated herein by reference. Prior to reshaping the chamber geometry, the heart walls experience high stress due to a combination of both the relatively large increased diameter of the chamber and the thinning of the chamber wall. Filling pressures and systolic pressures are typically high as well, further increasing wall stress. Geometric reshaping reduces the stress in the walls of the heart chamber to increase the heart's pumping efficiency, as well as to stop further dilatation of the heart.

Although the methods and devices are discussed hereinafter in connection with their use in the left ventricle and for the mitral valve of the heart, these methods and devices may be used in other chambers and for other valves of the heart for similar purposes. One of ordinary skill in the art would understand that the use of the devices and methods described herein also could be employed in other chambers and for other valves of the heart. The left ventricle and the mitral valve have been selected for illustrative purposes because a large number of the disorders occur in the left ventricle and in connection with the mitral valve.

The following detailed description of exemplary embodiments of the present invention is made with reference to the drawings, in which similar elements in different drawings are numbered the same. The drawings, which are not necessarily to scale, depict illustrative embodiments and are not intended to limit the scope of the invention.

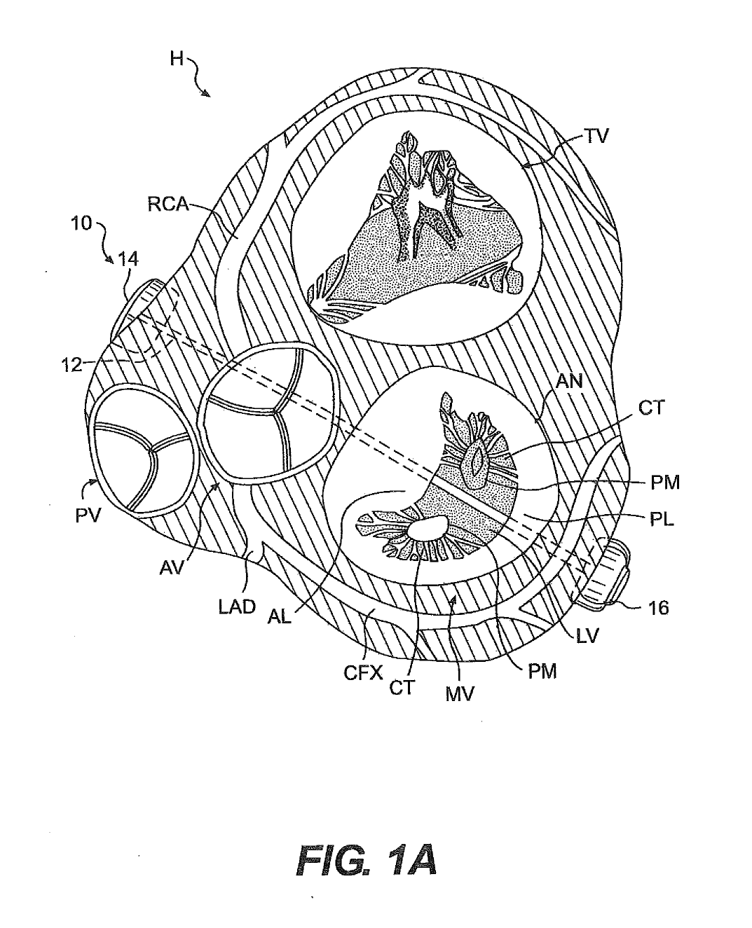

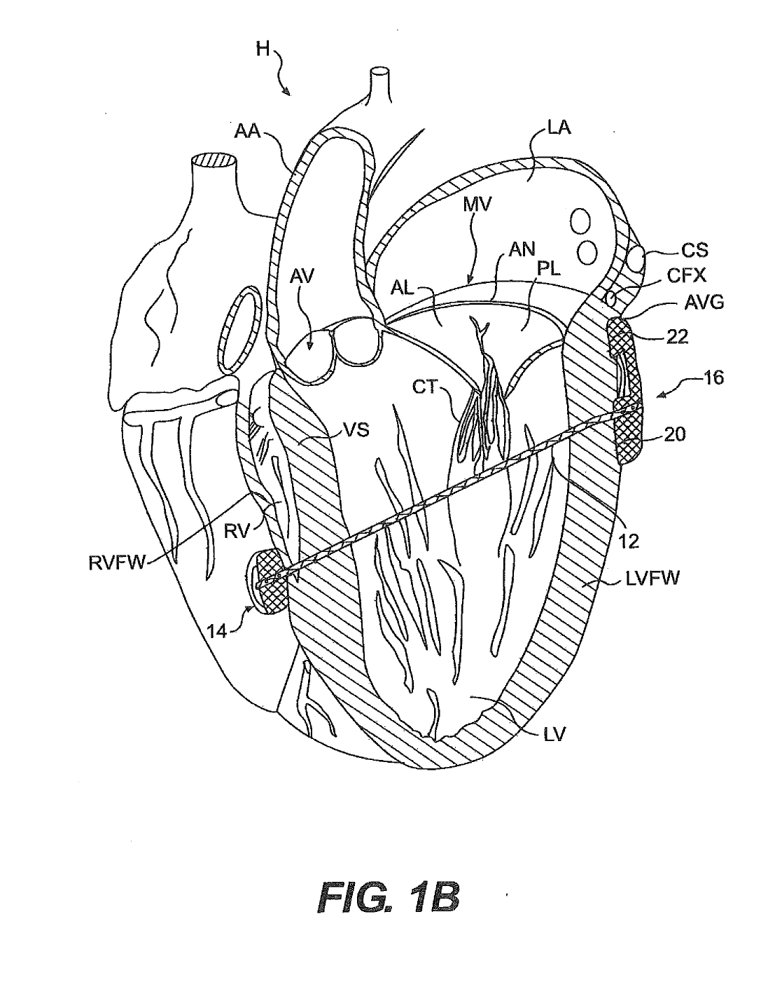

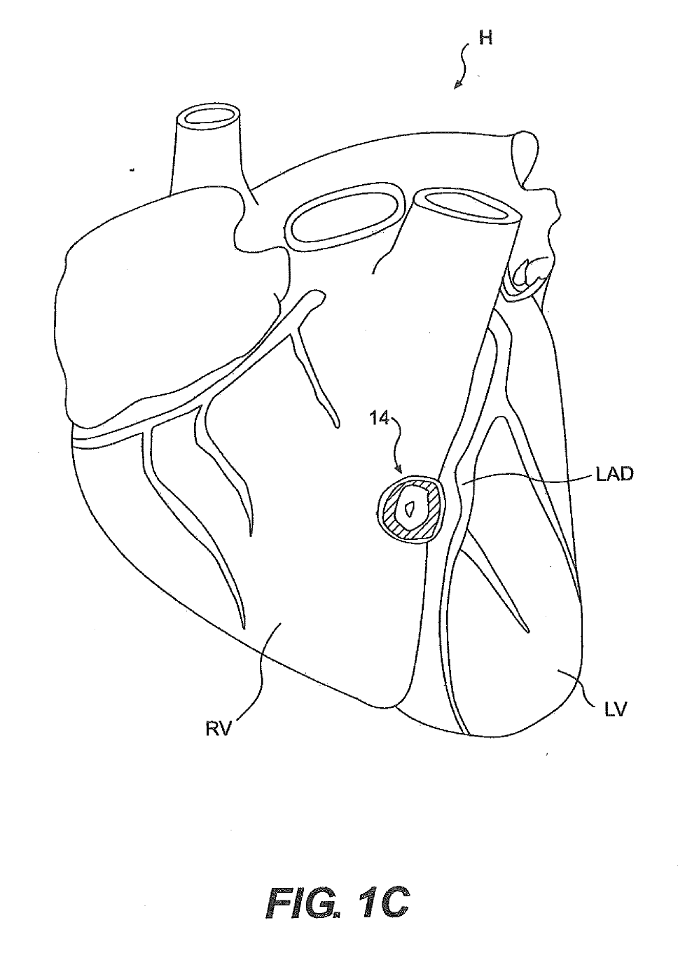

With reference to FIGS. 1A, 1B and 1C, a human heart H is shown during diastole. The devices and methods described herein are discussed with reference to the human heart H, but may also be applied to other animal hearts not specifically mentioned herein. A superior, short axis, cross-sectional view of the heart H is shown in FIG. 1A, a lateral, long axis, cross-sectional view of the human heart H is shown in FIG. 1B, and an anterior, long axis view of the human heart H is shown in FIG. 1C. In FIGS. 1A-1C, a mitral valve splint 10 is shown, which generally includes an elongate tension member 12 secured to an anterior pad 14 and a posterior pad 16.

For purposes of discussion and illustration, several anatomical features of the human heart are labeled as follows: left ventricle LV; right ventricle RV; left atrium LA; ventricular septum VS; right ventricular free wall RVFW; left ventricular free wall LVFW; atrioventricular groove AVG; mitral valve MV; tricuspid valve TV; aortic valve AV; pulmonary valve PV; papillary muscle PM; chordae tendeneae CT (or simply chordae); anterior leaflet AL; posterior leaflet PL; annulus AN; ascending aorta AA; coronary sinus CS; right coronary artery RCA; left anterior descending artery LAD; and circumflex artery CFX.

FIGS. 1A and 1B illustrate the mitral valve splint 10 extending through the heart H. As seen in FIG. 1A, the splint 10 substantially bisects the projection of the opening of the mitral valve MV and is aligned generally orthogonal to the arcuate opening defined between the anterior leaflet AL and posterior leaflet PL of the mitral valve MV. As seen in FIG. 1B, the splint 10 extends across the left ventricle LV at an inferior angle from the superior aspect of the left ventricular free wall LVFW, through the ventricular septum VS, and across the right ventricle RV near the intersection of the right ventricle RV and ventricular septum VS.

Both the anterior pad 14 and the posterior pad 16 are seated on the epicardium, while the tension member 12 extends through the myocardium and the ventricular chamber(s). This position also allows for the mitral valve splint 10 to have both pads 14, 16 placed epicardially, avoiding the need to position a pad interior to any of the heart chambers. To avoid interference with mitral valve MV function, the pads 14, 16 may be positioned such that the tension member 12 extends inferiorly of the of the leaflets AL/PL and chordae CT of the mitral valve MV. To maximize shape change effects of the mitral valve MV, and in particular the papillary muscles PM and/or annulus AN, the posterior pad 16 may have an inferior contact zone 20 and a superior contact zone 22, positioned on the epicardial surface proximate the papillary muscles PM and annulus AN, respectively.

The posterior pad 16 may be positioned such that the superior contact zone 22 rests in, or proximate to, the atrioventricular groove AVG, which is adjacent the annulus AN of the mitral valve MV. In this position, the application of deforming forces brought about by the posterior pad 16 causes a direct deformation of the annulus AN of the mitral valve MV, and/or repositioning of the papillary muscles PM. Both of these actions contribute to better coaptation of the leaflets AL, PL, minimizing or eliminating mitral valve regurgitation.

The anterior pad 14 may be positioned on the epicardial surface of the right ventricle RV, proximate the base of the right ventricular outflow track, and close to the intersection of the right ventricular free wall RVFW and the interventricular septum VS. In this position, the function of the right ventricle is minimally impacted when the splint 10 is tightened. Also in this position, the anterior pad 14 avoids interference with important blood vessels as well as important conduction pathways. For example, as seen in FIG. 1C, the anterior pad 14 may be so positioned to one side of the left anterior descending coronary artery LAD to avoid interference therewith.

The position of the splint 10 as shown in FIGS. 1A and 1B is exemplary, and it is anticipated that the position of the splint 10 may be virtually any orientation relative to the mitral valve MV leaflets AL, PL, depending on the heart failure and mitral valve regurgitation associated with the particular heart at issue. It is also contemplated that the mitral valve splint 10 may be utilized in conjunction with additional ventricular shape change devices such as those described in U.S. Pat. No. 6,261,222 to Schweich, Jr., et al., and/or U.S. Pat. No. 6,183,411 to Mortier, et al., the entire disclosures of which are incorporated herein by reference.

The mitral valve splint 10 may improve mitral valve function through a combination of effects. First, the shape of the annulus AN is directly altered, preferably during the entire cardiac cycle, thereby reducing the annular cross sectional area and bringing the posterior leaflet PL in closer apposition to the anterior leaflet AL. Second, the position and rotational configuration of the papillary muscles PM and surrounding areas of the left ventricle LV are further altered by the tightening of the splint 10. This places the chordae CT in a more favorable state of tension, allowing the leaflets AL, PL to more fully appose each other. Third, since the annulus AN of the mitral valve MV is muscular and actively contracts during systole, changing the shape of the annulus AN will also reduce the radius of curvature of at least portions of the annulus AN, just as the shape change induced by ventricular splints discussed hereinbefore reduces the radius of at least significant portions of the ventricle. This shape change and radius reduction of the annulus AN causes off-loading of some of the wall stress on the annulus AN. This, in turn, assists the annulus's ability to contract to a smaller size, thereby facilitating full closure of the mitral valve MV during systole.

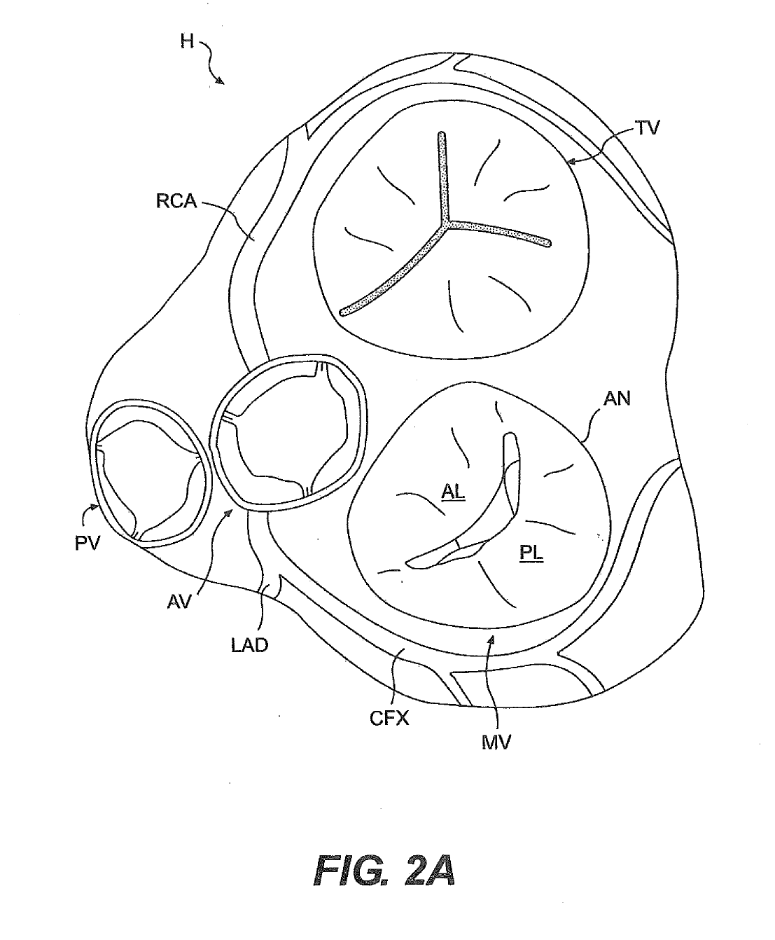

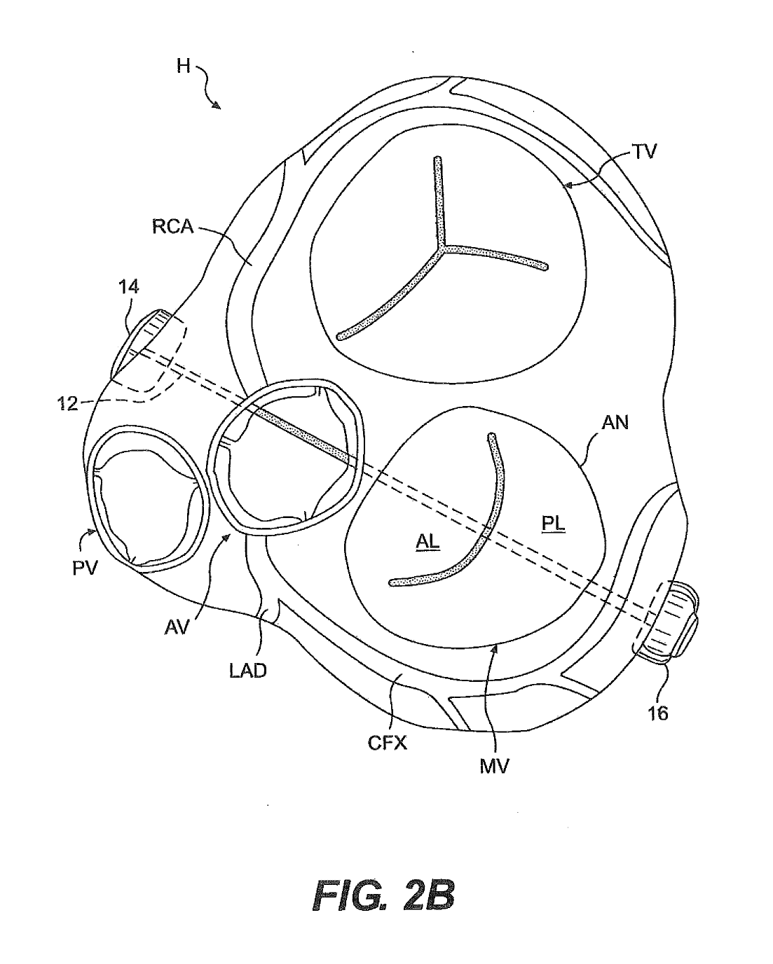

These effects are illustrated in FIGS. 2A and 2B. FIG. 2A shows an incompetent mitral valve MV during systole. The mitral valve MV is rendered incompetent by, for example, a dilated valve annulus AN. The mitral valve MV may become incompetent by several different mechanisms including, for example, a dilated valve annulus AN as mentioned above, or a displaced papillary muscle PM due to ventricular dilation. FIG. 2B shows the formerly incompetent mitral valve MV of FIG. 2A during systole as corrected with a mitral valve splint 10. As seen in FIG. 2B, the splint 10 causes inward displacement of a specific portion of the left ventricular free wall LVFW, resulting in a re-configuration and re-shaping of the annulus AN and/or the papillary muscles PM, thus providing more complete closure of the mitral valve leaflets AL, PL during systole.

As mentioned hereinbefore, the mitral valve splint 10 generally includes an elongate tension member 12 secured to an anterior pad or anchor 14 and a posterior pad or anchor 16. The pads 14, 16 may essentially function as epicardial anchors that engage the heart wall, do not penetrate the heart wall, and provide surfaces adjacent the exterior of the heart wall to which the tension member 12 is connected.

Tension member 12 may comprise a composite structure including an inner cable to provide mechanical integrity and an outer covering to provide biocompatibility. By way of example, not limitation, the inner cable of tension member 12 may have a braided-cable construction such as a multifilar braided polymeric construction. In general, the filaments forming the inner cable of the tension member 12 may comprise high performance fibers. For example, the inner cable may comprise filaments of ultra high molecular weight polyethylene available under the trade names Spectra™ and Dyneema™, or the inner cable may comprise filaments of some other suitable material such as polyester available under the trade name Dacron™ or liquid crystal polymer available under the trade name Vectran™.

The filaments forming the inner cable may be combined in yarn bundles of approximately 50 individual filaments, with each yarn bundle being approximately 180 denier. For example, two bundles may be paired together (referred to as 2-ply) and then braided with approximately 16 total bundle pairs to form the inner cable. The braided cable may include, for example, approximately 20 to 50 picks per inch (number of linear yarn overlaps per inch), such as approximately 30 picks per inch. The inner cable may have an average diameter of approximately 0.030 to 0.080 inches, for example, or approximately 0.055 inches, with approximately 1600 individual filaments. Further aspects of the inner cable of the tension member 12 are described in U.S. patent Ser. No. 09/532,049, now U.S. Pat. No. 6,537,198, filed Mar. 21, 2000, entitled A SPLINT ASSEMBLY FOR IMPROVING CARDIAC FUNCTION IN HEARTS, AND METHOD FOR IMPLANTING THE SPLINT ASSEMBLY (hereinafter referred to as the “049 patent application”), the entire disclosure of which is incorporated herein by reference.

When formed within the parameters indicated above, the inner cable permits the tension member 12 to withstand the cyclical stresses occurring within the heart chamber without breaking or weakening; provides a strong connection to the pads 14, 16; minimizes damage to internal vascular structure and the heart tissue; and minimizes the obstruction of blood flow within the heart chamber. Although exemplary parameters for the inner cable of the tension member 12 have been described above, it is contemplated that other combinations of material, yarn density, number of bundles, and pick count may be used, so as to achieve one or all the desired characteristics noted above.

The outer covering surrounding the inner cable of the tension member 12 may provide properties that facilitate sustained implantation in the heart. In particular, because tension member 12 may be in blood contact as it resides within a chamber of the heart H, the outer covering provides resistance to thrombus generation. Furthermore, because of the relative motion that occurs between the heart H and certain portions of tension member 12 passing through the heart chamber walls, the covering allows for tissue ingrowth to establish a relatively firm bond between the tension member 12 and the heart wall, thus reducing relative motion therebetween and minimizing potential irritation of the heart wall.

The outer covering surrounding the inner cable of the tension member 12 may be made of a porous expanded polytetrafluoroethylene (ePTFE) sleeve. The ePTFE material is biostable and tends not to degrade or corrode in the body. The ePTFE sleeve may have an inner diameter of approximately 0.040 inches and a wall thickness of approximately 0.005 inches, for example, prior to placement around the inner cable of the tension member 12. The inner diameter of covering may stretch to fit around the inner cable to provide a frictional fit therebetween. The ePTFE material of the covering may have an internodal distance of between approximately 20 and approximately 70 microns, such as approximately 45 microns, for example. This may permit cellular infiltration and thus result in secure ingrowth of the adjacent heart wall tissue so as to create a tissue surface on the tension member 12 residing in the heart chamber. The ePTFE material, particularly having the internodal spacing discussed above, has a high resistance to thrombus formation and withstands the cyclic bending environment occurring in the heart. Further aspects of the outer covering of the tension member 12 are described in the '049 patent application. Although ePTFE has been described as a suitable material for the outer covering of the tension member 12, other suitable materials exhibiting similar characteristics may also be used.

The anterior pad 14 and the posterior pad 16 of the mitral valve splint 10 are connected to opposite ends of the tension member 12. To facilitate delivery of the splint 10 as described in more detail hereinafter, one of the anchor pads 14, 16 may be fixed and locked to the tension member 12 prior to implantation. The other of the anchor pads 14, 16 may be initially adjustable and subsequently fixed to the tension member 12. In particular, its position along the length of the tension member 12 may be adjusted during implantation, prior to fixation to the tension member 12. The posterior pad 16 may be positioned proximate the posterior leaflet PL of the mitral valve MV and may be fixed relative to tension member 12. The anterior pad 14 may be positioned near the intersection of the right ventricle RV and ventricular septum VS, and may be initially adjustable relative to tension member 12 and subsequently fixed thereto.

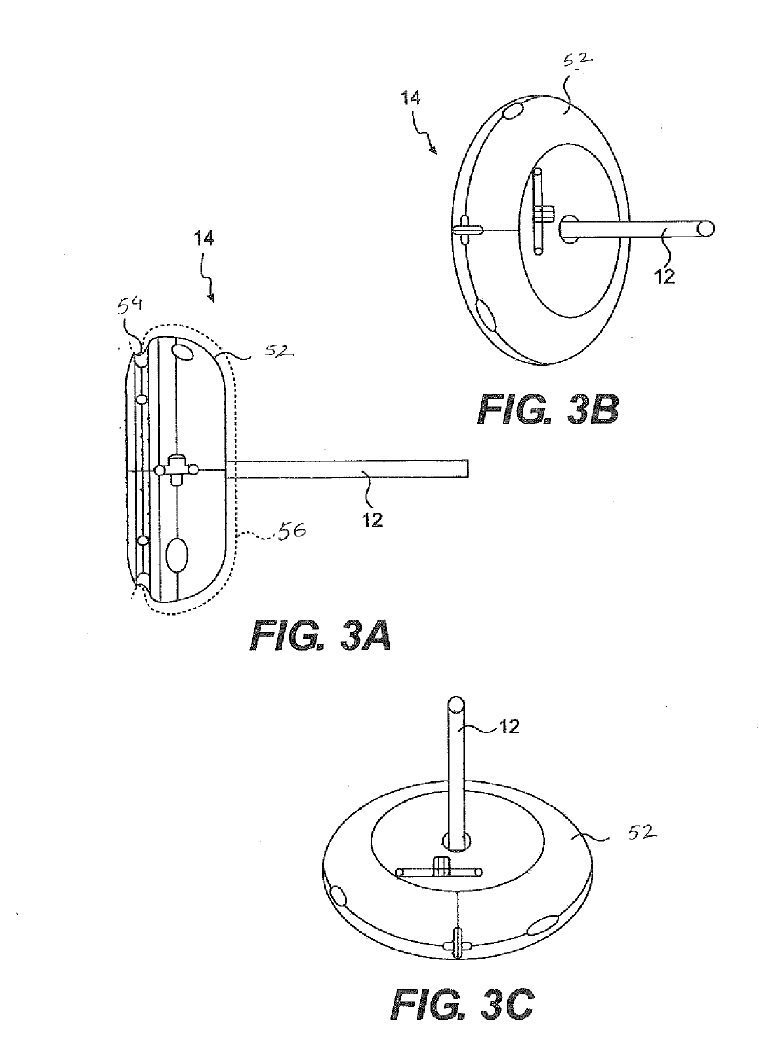

In the exemplary embodiments described herein, the anterior pad 14 is an adjustable pad, but may be fixed as well. The anterior pad 14 may have a substantially circular shape as shown in FIG. 1C or an oval shape as shown in FIGS. 3A-3C. The oval shape of the anterior pad 14 increases the contact surface area relative to the circular shape in order to more effectively match the contact surface area of the posterior pad 16. This serves to balance the deformations and contact stresses brought about by each pad 14/16.

With reference to FIGS. 3A-3C, an oval shaped anterior pad 14 is shown. The anterior pad 14 may include a convex inner surface 52 that engages the epicardium when the splint 10 is implanted in the heart H. The anterior pad 14 also includes a circumferential groove 54 to accommodate suture windings to secure a pad covering 56 (shown in phantom). The pad covering 56 may be made of a velour woven polyester material, for example, available under the trade name Dacron™, or other similar suitable material such as expanded polytetrafluoroethylene (ePTFE). The pad covering facilitates ingrowth of the heart wall tissue to secure the pad to the epicardium and thereby prevent long-term, motion-induced irritation thereto. The anterior pad 14 further includes a plurality of inner components (e.g., pins) and channels (not visible) to permit adjustable fixation of the pad 14 to the elongate tension member 12. These features and further aspects of the anterior pad 14 are described in the '049 patent application.

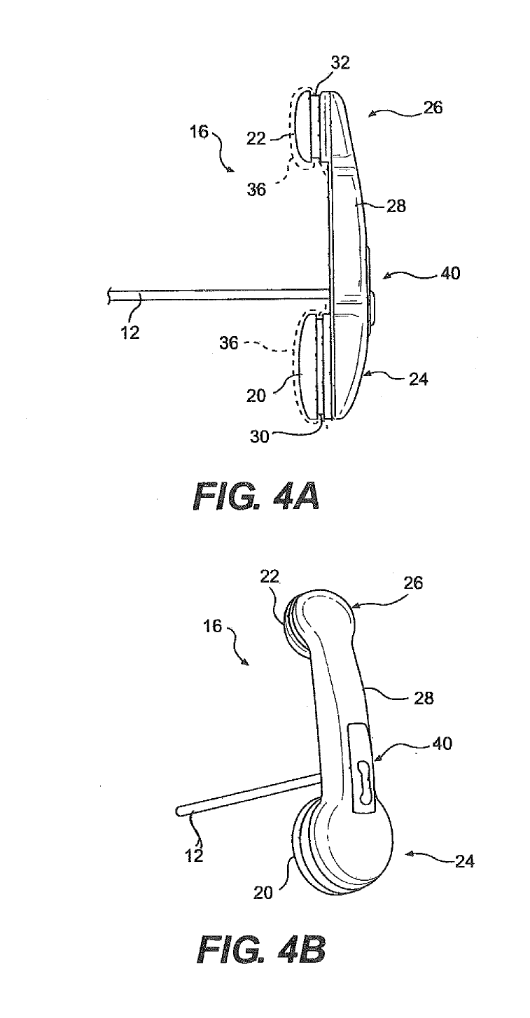

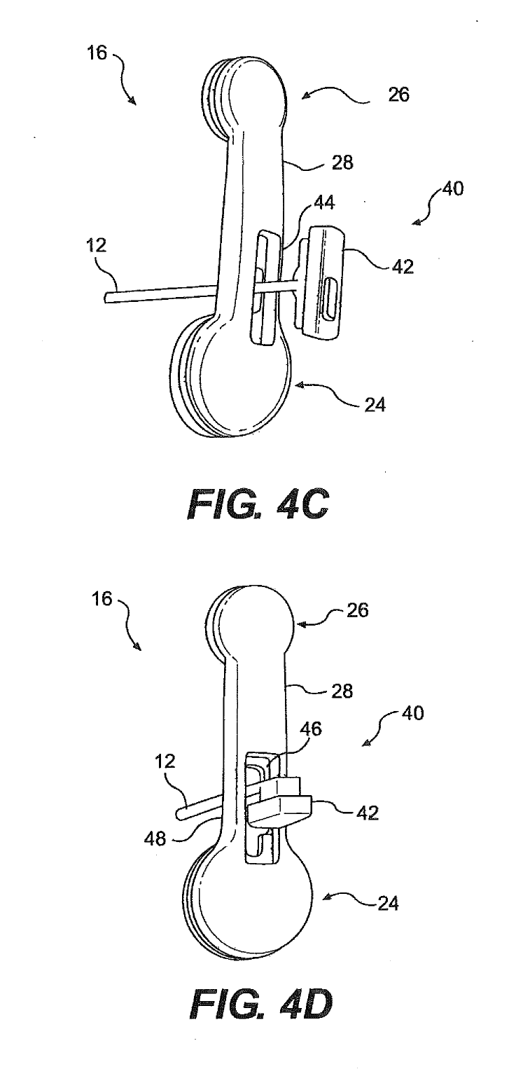

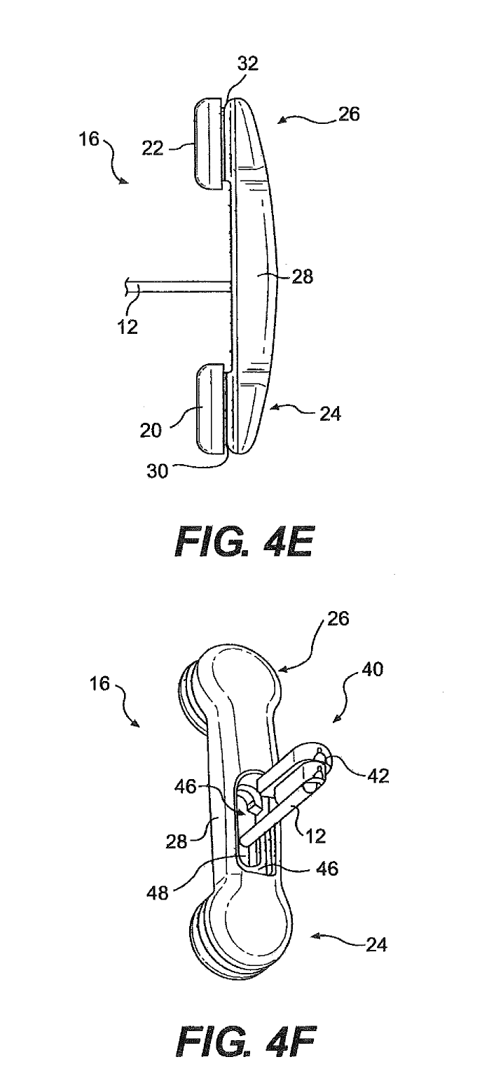

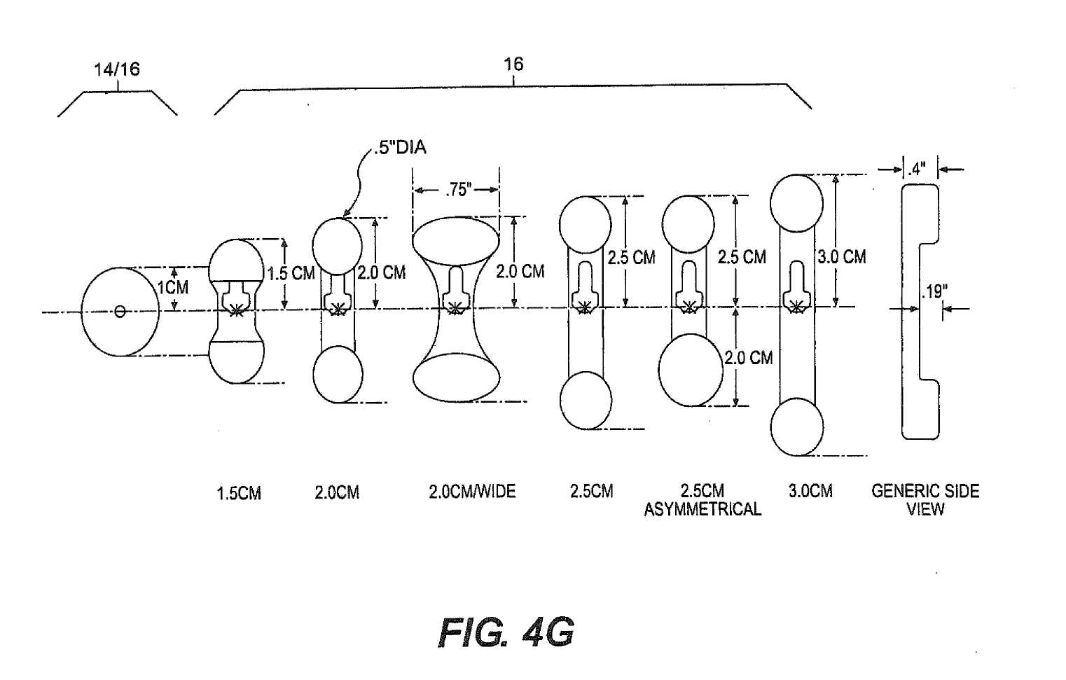

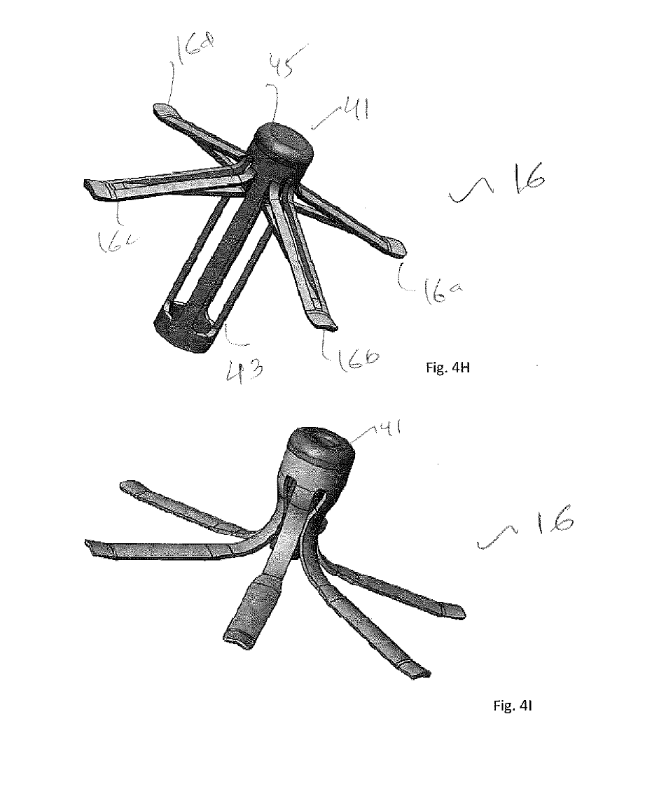

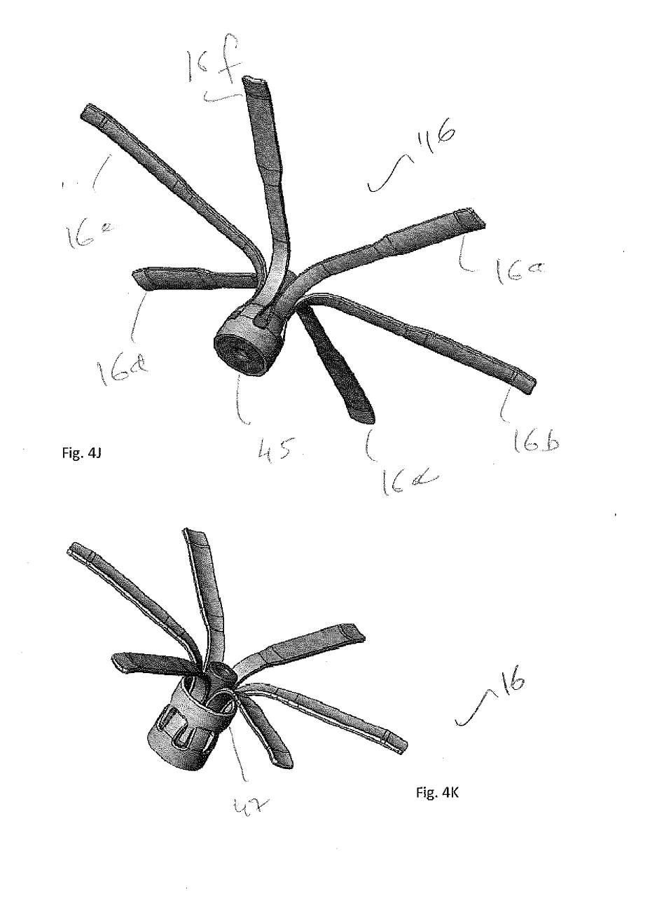

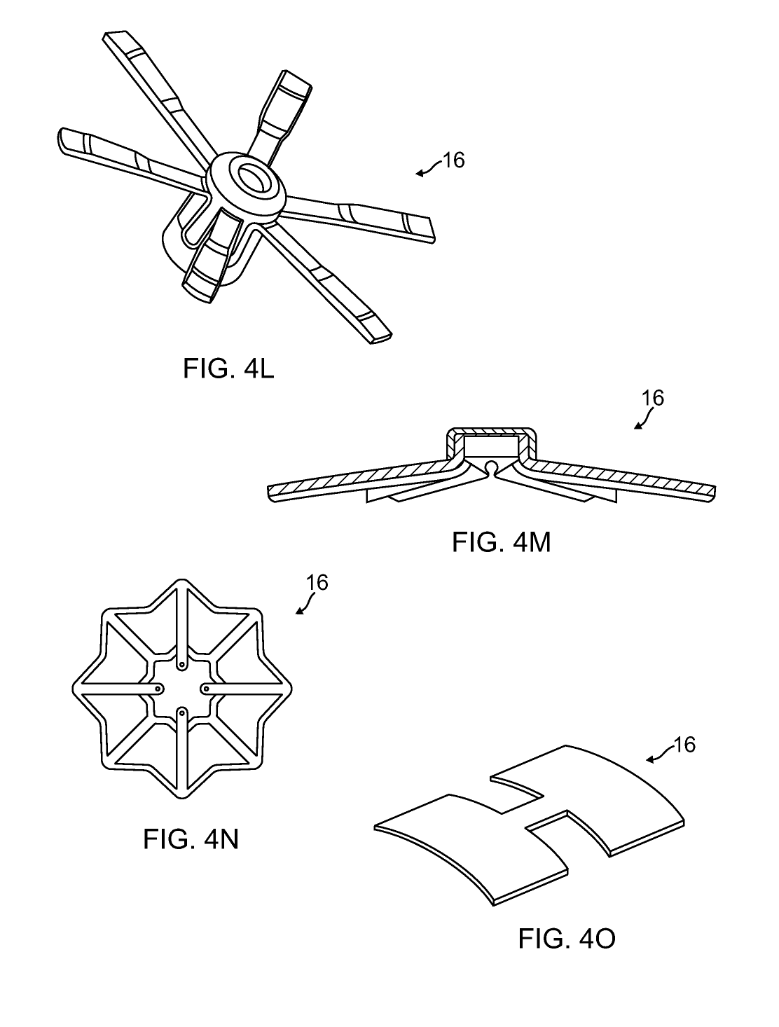

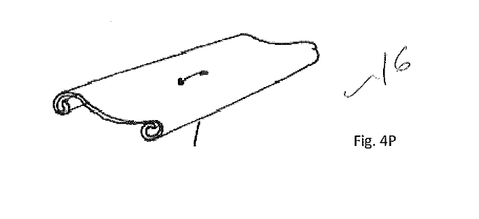

With reference to FIGS. 4A-4F, a posterior pad 16 of the mitral valve splint 10 is shown. In the exemplary embodiments described herein, the posterior pad 16 is a fixed pad, but may be adjustable as well. The posterior pad 16 may define one, two or more contact zones. For example, the posterior pad 16 may define a superior contact zone 22 and an inferior contact zone 20 connected therebetween by bridge 28. The superior contact zone 22 may rest on the epicardial surface of the left ventricle LV, adjacent the annulus AN of the mitral valve MV associated with the posterior leaflet PL. The inferior contact zone 20 may rest on the epicardial surface near the level of the papillary muscles PM of the mitral valve MV, positioned, for example, midway between the papillary muscles PM.

The tension member 12 may intersect the bridge 28 of the posterior pad 16 closer to the inferior end 24 than the superior end 26 as seen in FIG. 4A, for example. The pad 16 thus serves to provide a deformation of a superior portion of the left ventricle LV adjacent the annulus AN of the mitral valve MV, while allowing the tension member 12 to connect to the pad 16 at a position low enough to minimize interference between the tension member 12 and the mitral valve MV structures. To balance the longer moment arm of the bridge 28 exerted by the superior contact zone, the inferior contact zone may have a larger epicardial contact area.

Other posterior pad 16 shapes and sizes are also contemplated, possessing varying numbers and positions of contact zones, possessing varying distances between the contact zones and the tension member, and possessing varying shapes and sizes of contact zones. For example, as shown in FIGS. 4E and 4F, the tension member may alternatively intersect the bridge 28 midway between the superior end 26 and the inferior end 24, and the superior and inferior contact zones 22, 20 may have equal contact surface areas. As a further alternative, the posterior pad 16 may be relatively small, and not necessarily elongated, with the tension member 12 connected to the center of the pad 16 (similar to anterior pad 14), such that the position of the tension member 12 relative to the mitral valve structure is slightly elevated as compared to the embodiment illustrated. In a specific embodiment, the posterior pad may be dumbbell shaped. Exemplary dimensions and shapes of posterior pad 16 are illustrated in FIG. 4G.

In addition to variations of the design of posterior pad 16, it is also contemplated that variables associated with the position of the pad 16 and forces applied to the pad 16 by the tension member 12 may be selected as a function of, for example, the particular manifestation of mitral valve dysfunction and/or as a function of the particular anatomical features of the patient's heart. These variables may affect the magnitude, area, and/or specific location of displacement of the left ventricular free wall LVFW proximate the mitral valve MV structures (annulus AN, leaflets AL/PL, chordae CT, and/or papillary muscles PM).

With continued reference to FIGS. 4A-4G, the contact zones 20, 22 may have a convex surface that engages the epicardium when the splint 10 is implanted in the heart H. The posterior pad 16 also includes circumferential grooves 30, 32 on each of the contact zones 20, 22 to accommodate suture windings to secure a pad covering 36 (shown in phantom). The pad covering 36 may be made of the same or similar material discussed hereinbefore with reference to anterior pad 14, to facilitate tissue in-growth after implantation.

The posterior pad 16 may incorporate a releasable connection mechanism 40 that allows the pad 16 to be removed from the elongate tension member 12 and replaced, for example, by a different pad with an alternate shape and size, depending on the particular anatomy of the heart H and/or the desired effects on the heart. It may be desirable, for example, to utilize a pad 16 that has a longer bridge 28 with greater spacing between the contact zones 20, 22 to minimize mitral regurgitation (MR). Although the connection mechanism 40 allows the pad 16 to be removed from the tension member 12 and replaced with another pad 16, the position of the pad 16 may remain fixed in that the final position of the pad 16 along the linear aspect of the tension member 12 is fixed, as opposed to the adjustable anterior pad 14 discussed hereinbefore.

The releasable connection mechanism 40 may comprise a block 42 which fits into a recessed region 44 within the pad bridge 28, as best seen in FIGS. 4C and 4F. The block 42 may be fixed to the tension member by one or more pins that penetrate the braided inner cable of the tension member 12, in a manner similar to the connection of the tension member 12 to the anterior pad 14. The recessed region 44 may have a length, width, and height corresponding to the length, width, and height of the block 42, respectively. As best seen in FIGS. 4D and 4F, an inwardly projecting rim 46 is provided at the bottom of the recessed region 44, which prevents the block 42 from moving through the pad bridge 28 in response to tension forces exerted by the tension member 12. An opening 48 is defined by the edge of the rim 46 and is sized such that the block 42 may be passed through the bridge 28 of the pad 16 when the block 42 is lifted away from the bridge 28 and rotated as shown in FIGS. 4D and 4F. A different pad 16, having perhaps a different shape and/or dimensions, may then be connected to the block 42 and tension member 12 by reversing the steps discussed above before final implantation of the splint 10.

FIGS. 4H-4P show alternate embodiments of the posterior pad of the mitral valve 10. In the exemplary embodiments described herein, the posterior pad 16 is a fixed pad, but may be adjustable as well. As a further alternative, as shown in FIGS. 4H-4P, the posterior pad 16 may be relatively small, and not necessarily elongated, with the tension member 12 connected to the center of the pad 16 (similar to anterior pad 14), such that the position of the tension member 12 relative to the mitral valve structure is slightly elevated as compared to the embodiment illustrated.

As illustrated in FIG. 4H, the posterior pad 16 has a plurality of integral legs 16a-16d connected to a retaining mechanism. The integral legs 16a-16d extend at an angle from the central end of the retaining member. The retaining member comprises a retaining chamber 43, and a locking member 45. Retaining chamber 43 is a tubular structure for retaining the tension member 12 therein, and the locking member 45 anchors the tension member with the pad 16.

In an alternate embodiment illustrated in FIGS. 41-4L, the retaining chamber 43 is formed by the tapering of the integral legs 16a-16f. As shown in FIGS. 4K and 4L, the retaining mechanism 41 may also include a sleeve positioned beneath the locking element 45, for further securing the splint 10 within the retaining chamber 43. FIGS. 4H-4P illustrate different shapes of anchoring pads 16 according to one embodiment.

It is important to note that while an exemplary embodiment of a mitral valve splint 10 is described above, variations are also considered within the scope of the disclosure. Mitral valve and cardiac anatomy may be quite variable from patient to patient, and the mitral valve splint design and implant position may vary accordingly. For example, the location of the regurgitant jet may be centered, as shown in FIG. 2A, or may favor one side of the valve opening. Therefore, differences in posterior pad size, pad shape, and overall splint location, for example, may be required to best modify the heart chamber and valve annulus for a particular patient. Steps taken during the delivery of the mitral valve splint 10 are useful to identify and incorporate these designs and position variables to suit the particular cardiac anatomy and mitral valve dysfunction.

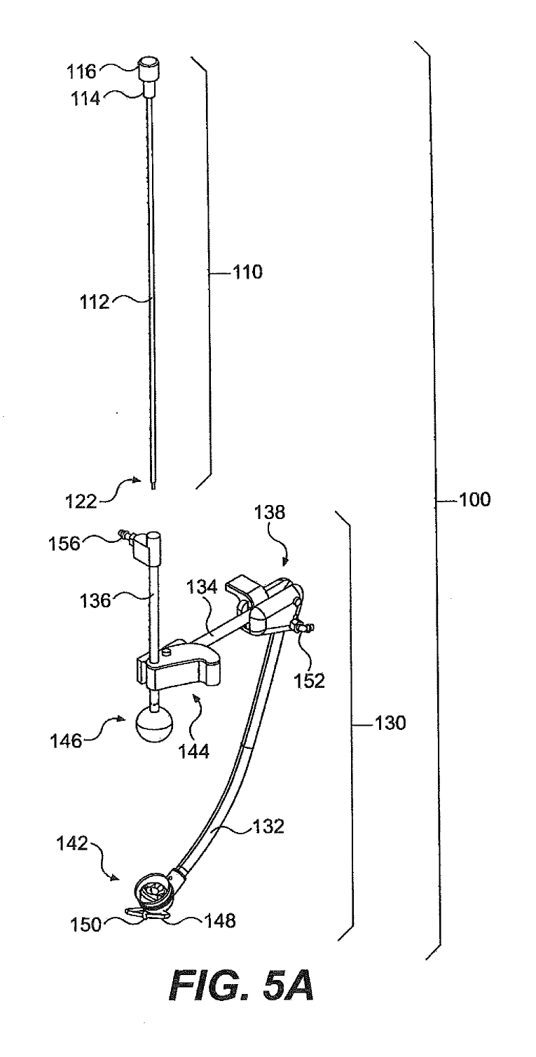

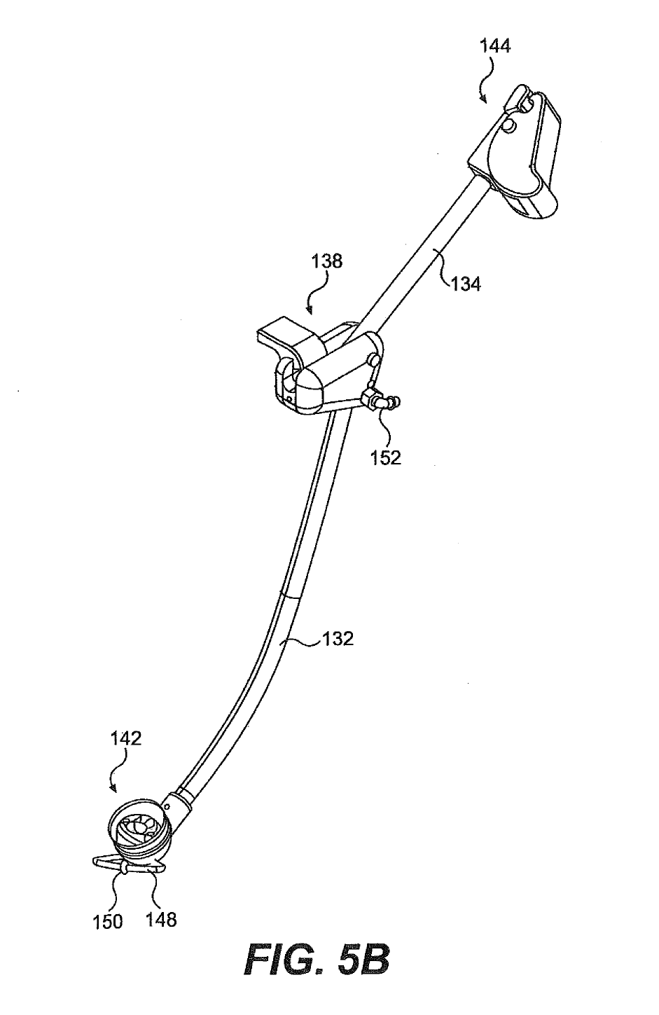

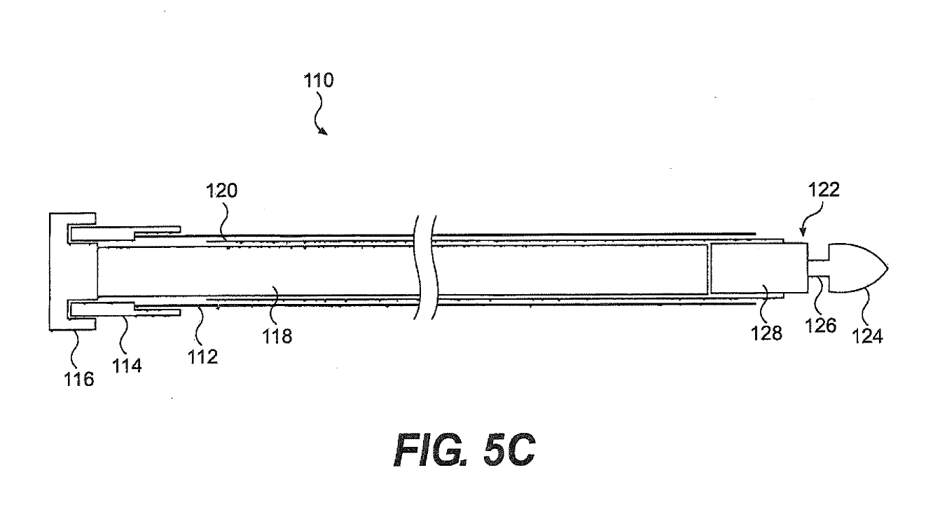

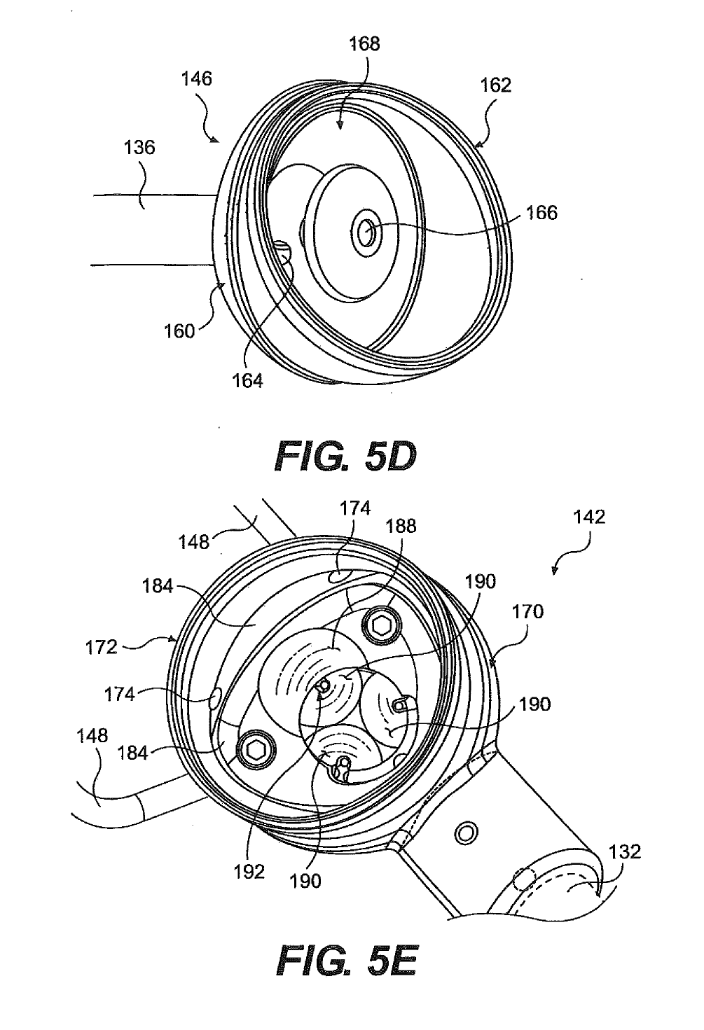

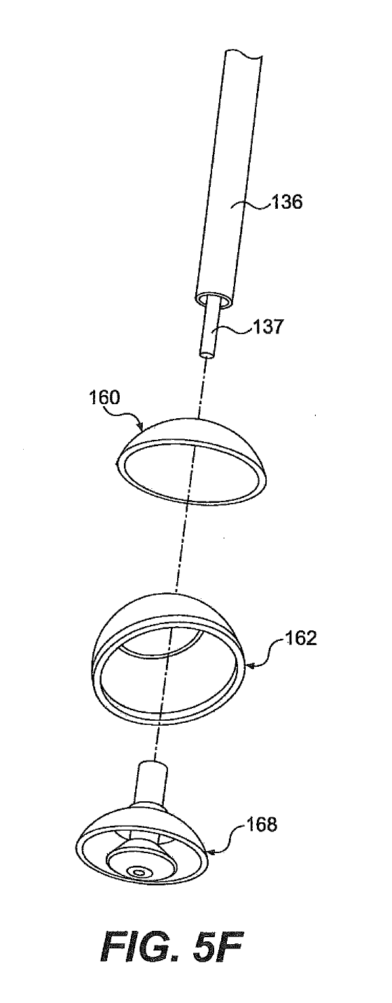

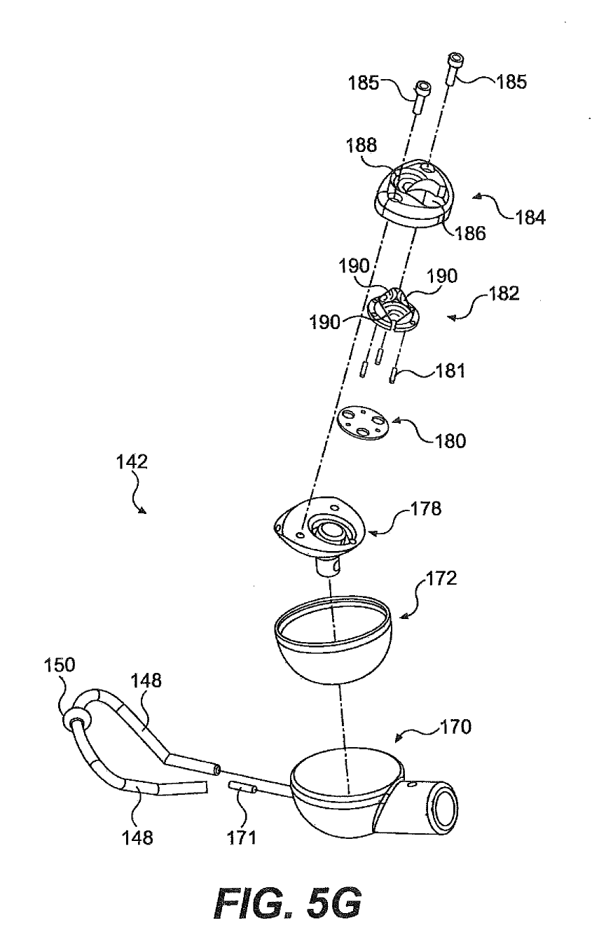

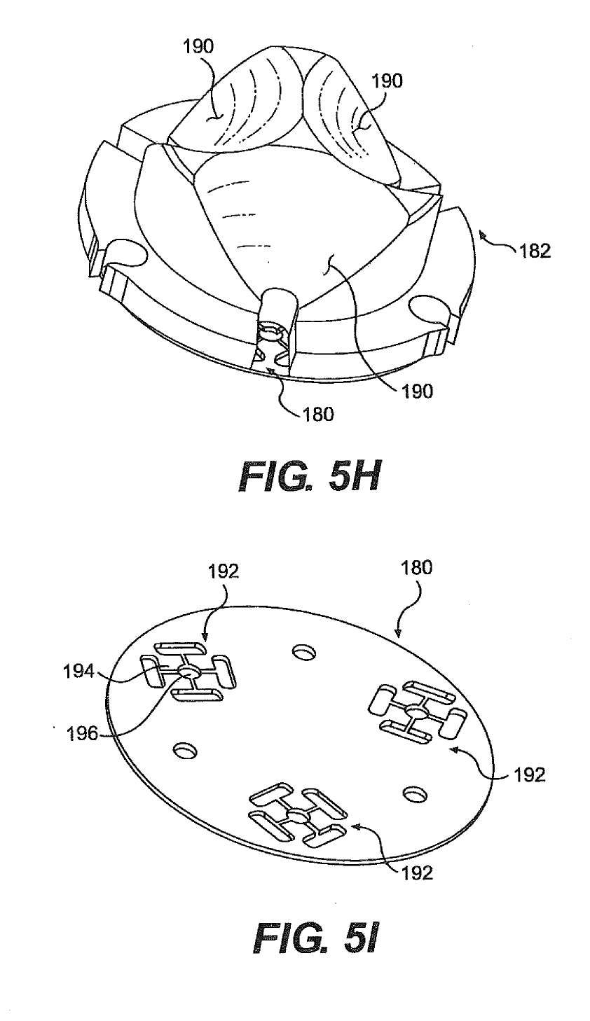

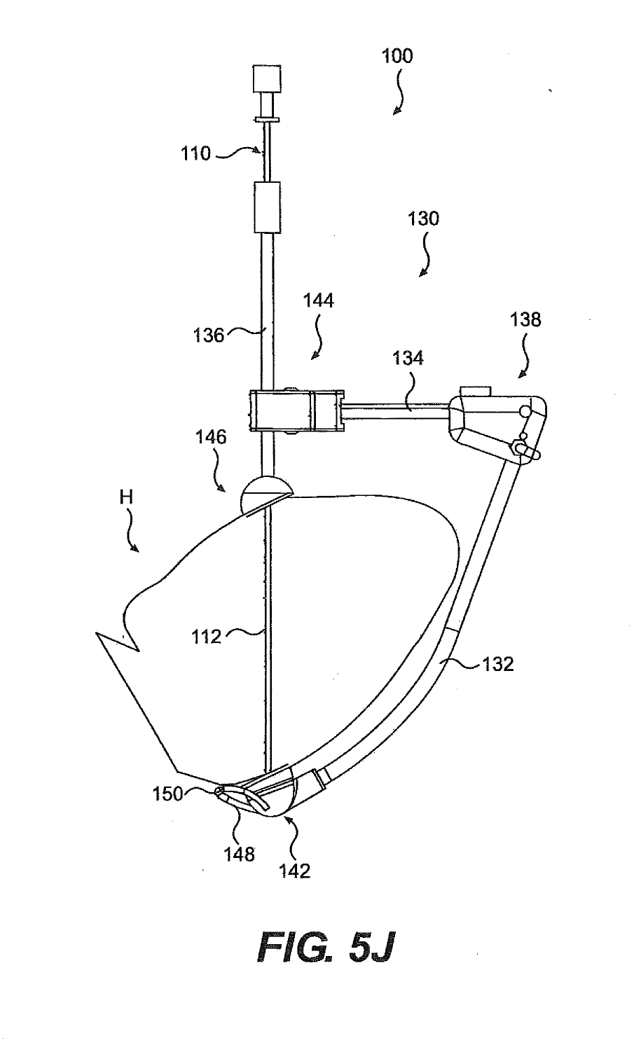

With reference to FIG. 5A, a mitral valve splint delivery system 100 is shown. The mitral valve splint delivery system 100 and associated methods are exemplary, non-limiting embodiments for the delivery of mitral valve splint 10. The mitral valve splint delivery system 100 may include a needle delivery assembly 110, in addition to a positioning and alignment device 130. The positioning and alignment device 130 may be used for identifying and maintaining the desired positions for the subsequent placement of the posterior pad 16 and the anterior pad 14, and the needle delivery assembly 110 may be used for passing the tension member 12 of the splint 10 through the heart H.

The positioning and alignment device 130 may include a posterior arm 132, a swing arm 134, and an anterior arm 136. A lockable hinge 138 allows for relative planar rotation between the posterior arm 132 and the combination of the swing arm 134 and the anterior arm 136. The “closed” position of the hinge 138 is shown in FIG. 5A, and the “open” position of the hinge 138 is illustrated in FIG. 5B. The anterior arm 136 may be joined to the swing arm 134 via a releasable securing clamp 144.

The posterior arm 132 and the anterior arm 136 each may have associated vacuum chambers 142, 146, respectively, for temporarily securing the positioning and alignment device 130 to the epicardial surface of the heart H. At a predetermined spacing from the posterior vacuum chamber 142, an indicator ball 150 may be connected thereto by a fixed dual-arm member 148. The anterior arm 136 may contain a tube defining a lumen for passage of the needle delivery assembly 110 therethrough. The anterior arm 136 and the posterior arm 132 each may have an associated vacuum lumen (not visible) extending therethrough in fluid communication with their respective vacuum chambers 146, 142. Associated fittings 156, 152 may be provided on the anterior arm 136 and the posterior arm 132, respectively, for connecting the corresponding vacuum lumens to a vacuum source (not shown).

With reference to FIG. 5C, the needle delivery assembly 110 may include an outer tube 112, which may be formed of a relatively rigid material such as, for example, a metal (e.g., stainless steel). Other suitable materials also may be used for the outer tube 112. The proximal end of the outer tube 112 may be fixedly connected to a hollow base 114 which may be fixedly or releasably connected to a cap 116. The cap 116 may be fixedly connected to a core member 118 which extends through the outer tube 112 and which may be formed of a relatively rigid material such as, for example, a metal (e.g., stainless steel). A guide tube 120 may be disposed between the outer tube 112 and the inner core member 118. The guide tube 120 may be relatively flexible, kink resistant, and lubricious. For example, the guide tube 120 may be formed of a PTFE liner covered by a metallic braid with a thermoplastic covering such as Nylon. Other suitable materials that permit the guide tube to be relatively flexible, kink resistant, and lubricious also may be used. A tip member 122 including, for example, a sharpened spearhead or bullet-shaped end 124 may be fixedly connected to a distal portion of the guide tube 120 by swaging a short metal tube (not shown) over the guide tube 120 and onto a proximal portion 128 of the tip member 122.

With reference to FIGS. 5D and 5F, the anterior vacuum chamber 146 is shown. The anterior vacuum chamber 146 includes a base housing 160, an articulating rim 162 and a base cover 168. The articulating rim 162 is captured between base housing 160 and base cover 168. A proximal end of the base cover 168 and the base housing 160 are fixedly connected to the anterior arm 136. The articulating rim 162 is movable with respect to the base housing 160, base cover 168 and anterior arm 136, thus allowing the rim 162 to make good contact with the epicardial surface of the heart H and form an effective seal upon application of a vacuum.

In FIG. 5F, the needle tube 137 defining the needle lumen therein is visible extending through the anterior arm tube 136. The lumen of the needle tube 137 opens into the interior of the anterior vacuum chamber 146 at needle port 166. The annular vacuum lumen defined between the needle tube 137 and the anterior arm tube 136 opens into the interior of the anterior vacuum chamber 146 at vacuum port 164.

With reference to FIGS. 5E, 5G, 5H, and 5I, the posterior vacuum chamber 142 is shown. The posterior vacuum chamber 142 includes a base housing 170, an articulating rim 172 and a base cover 178. A proximal end of the base housing 170 is fixedly connected to the posterior arm 132, and the base cover 178 is secured to the base housing 170 by pin 171. The articulating rim 172 is captured between base housing 170 and base cover 178. The articulating rim 172 is movable with respect to the base housing 170, base cover 178 and posterior arm 132, thus allowing the rim 172 to make good contact with the epicardial surface of the heart H and form an effective seal upon application of a vacuum. The base cover 178 includes vacuum ports 174 which are in fluid communication with the interior of the posterior vacuum chamber 142 and which define a fluid path to the vacuum lumen in the posterior arm 132.

The posterior vacuum chamber 142 may include a retainer mechanism. For example, a capture plate 180 may be connected to a rotating insert 182 by connector pins 181. The capture plate 180 and rotating insert 182 are collectively captured between the base cover 178 and a capture plate cover 184, which is secured to the base cover 178 by screws 185. The capture plate 180 and rotating insert 182 are collectively rotatable relative to the base cover 178 and a capture plate cover 184.

The capture plate cover 184 defines an offset opening 186 into which the upper portion of the rotating insert 182 is positioned. The capture plate cover 184 also defines a semi-conical concave slope 188. Similarly, the rotating insert 182 defines a plurality of semi-conical concave slopes 190 that may be individually aligned with the slope 188 on the capture plate cover 184 by indexing (rotating) the rotating insert 182 relative to the capture plate cover 184 such that the semi-conical concave slopes 188, 190 collectively define a conical funnel that serves to guide the needle assembly 110 into the desired dock 192. Thus, if a needle assembly 110 is initially deployed in a first (center) dock 192, and it is desired to re-deploy another needle assembly 110, the rotating insert 182 and capture plate 180 may be collectively rotated relative to the capture plate cover 184 to align a second (auxiliary) dock 192 and its associated semi-conical slope 190 with the semi-conical slope 188 of the capture plate cover 184.

As seen in FIGS. 5H and 5I, the capture plate 180 is fixed to the bottom side of the rotating insert 182, with each dock 192 positioned at the bottom of the semi-conical slopes 190. Each dock 192 includes a plurality of deflectable retainer tabs 194 defining a central hole 196. The capture plate 180 may comprise a spring temper stainless steel and the docks 192 may be formed by selectively etching the plate using a photo-etch technique, for example.

As the bullet-shaped tip 124 of the needle assembly 110 is advanced into the posterior vacuum chamber 142, it is guided to a central dock 192 by the funnel collectively defined by slopes 188, 190. As the bullet-shaped tip 124 is advanced further into hole 196, the tabs 194 are resiliently deflected away. After the bullet-shaped tip 124 passes the tabs 194 and the distal end thereof is stopped by base cover 178, the tabs 194 resiliently spring back into the detent space 126 of the tip assembly 122, serving to lock the position of the tip assembly 122 and guide tube 120 relative to the posterior vacuum chamber 142.

Those skilled in the art will recognize that the positioning and alignment device 130 may be formed of a variety of materials and may have a variety of dimensions depending on, for example, the conditions of use and anatomical variability. By way of example, not limitation, the posterior arm 132, swing arm 134 and anterior arm 136 may be formed of stainless steel tubing. The connective elements (pins, screws, etc.) may also be formed of stainless steel. The rims 162, 172 of the anterior and posterior vacuum chambers 146, 142, respectively, may be formed of clear polycarbonate, or other similar suitable material, to facilitate visualization of the epicardial surface thereunder. The dual-arm 148 and the indicator ball 150 may be formed of PEEK with a stainless steel core wire running therethrough. The remaining components of the positioning and alignment device 130 may be formed of a polymeric material such as acetyl available under the trade name Delrin™. The vacuum lines connecting the fittings 152/156 to a vacuum source may comprises polyether block amide tubes with stainless steel coil windings therein. Other suitable materials may be used and are contemplated as being within the scope of the disclosure.

Also by way of example, not limitation, the posterior arm 132 may have a length of approximately 18 cm, the swing arm 134 may have a length of approximately 10 cm, and the anterior arm may have a length to accommodate approximately 5 cm to 13 cm of adjustable distance between the anterior vacuum chamber 146 and the posterior vacuum chamber 142. These exemplary dimensions have been found to accommodate a wide variety of anatomical sizes and variations. The needle assembly 110 may have a length of approximately 46 cm to traverse the heart H and provide sufficient length and flexibility for manipulation around the heart. The anterior vacuum chamber 146 and the posterior vacuum chamber 142 may have outside diameters of approximately 2 cm to provide adequate yet atraumatic holding power on the epicardium. Other suitable dimensions may be selected depending on a patient's particular anatomy, for example.

In use, the positioning and alignment device 130 is initially in the open position. The posterior arm 132 may be positioned through a thoracotomy (e.g. a median sternotomy), along the posterior aspect of the heart H and generally aligned with the long axis of the left ventricle LV. The indicator ball 150 may be positioned in the AV groove, by visual or tactile cues, or a combination of such cues. During this procedure, the heart H may be manipulated to facilitate direct visualization. The predetermined distance between the indicator ball 150 and the posterior vacuum chamber 142 places the vacuum chamber 142 in a desired position relative to the annulus AN of the mitral valve MV. The posterior vacuum chamber 142 is activated by applying a vacuum thereto, securing the chamber 142 to the epicardial wall is the desired position. The center of the posterior vacuum chamber 142 now corresponds to the future location of the intersection of the tension member 12 with the left ventricular LV chamber wall.

Assessment of the position of the posterior vacuum chamber 142 relative to internal mitral valve MV structures such as leaflets AL, PL, papillary muscles PM, and regurgitant jet may be performed with ultrasonic imaging such as trans-esophageal or epicardial echocardiography. The position of the posterior vacuum chamber 142 may be visualized on the echocardiogram by observing the portion of the left ventricular free wall LVFW that is less dynamic than the remaining portions thereof, rendered so by the dampening effect of the posterior vacuum chamber 142 fixed thereto. Mechanical manipulation of the positioning and alignment device 130 may also be performed to assess the functional impact of this position on the mitral valve regurgitation, as the heart is still beating. For example, the positioning and alignment device 130 may be pivoted about the posterior vacuum chamber 142 to drive the indicator ball 150 into the AV groove, thereby exerting an inward force on the annulus AN of the mitral valve MV. If the position is not optimal, the vacuum may be de-activated, and the posterior vacuum chamber 142 may be repositioned as desired. Conveniently, the posterior vacuum chamber 142 will leave a pucker mark on the epicardium at the initial position thereof, which may serve as a reference mark for repositioning.

The anterior arm 136, initially disconnected from the swing arm 134, is then manipulated to position the anterior vacuum chamber 146 on the epicardial surface of the heart, corresponding to the subsequent desired position of the anterior anchor pad 14. As the anterior arm is manipulated, echocardiographic information pertaining to the right ventricle RV and nearby tricuspid valve TV may be assessed and utilized to help find a desired position for the anterior vacuum chamber 146. Once in a desired position, the anterior vacuum chamber 146 is activated by application of vacuum, temporarily securing anterior vacuum chamber 146 to the epicardial surface of the heart. The swing arm 134 is then rotated into position to allow for the securing clamp 144 to clamp onto the anterior arm 136. The anterior arm 136 preferably is long enough (e.g., 5 to 15 cm) to allow for significant variations in heart diameters from patient to patient.

Both vacuum chambers 142, 146 are now securely positioned on the epicardial surface of the heart, in positions which will correspond to the anterior and posterior anchor pads 14, 16. The needle delivery assembly 110 now may be inserted through the passage lumen provided in the anterior arm 136, through the anterior vacuum chamber 146, across the heart and into the posterior vacuum chamber 142. The positioning and alignment device 130, with the needle delivery assembly 110 fully inserted through the heart chamber, is illustrated in FIG. 5J.

As the needle delivery assembly 110 is passed into the posterior vacuum chamber 142, the circumferential detent 126 on the tip assembly 122 engages with the retention mechanism of the posterior vacuum chamber 142. Once the needle delivery assembly 110 is locked in position in the central dock 192, the cap 116 and base 114 are pulled proximally from the anterior arm 136, thus removing the outer tube 112 and core member 118 from the needle delivery assembly 110. The tip assembly 122 and guide tube 120 are thus left in position across the heart chamber and define the path that will be taken by the tension member 12 through the heart H.

The vacuum to the anterior and posterior chambers 146, 142 may then be interrupted, allowing the positioning and aligning device 130 to be removed from the surface of the heart. As the positioning and aligning device 130 is removed from the heart, the tip assembly 122 and guide tube 120 remain engaged with the posterior vacuum chamber 142, bringing the tip assembly 122 and distal end of the guide tube 120 to an easily accessible location nearer the anterior side of the heart H. The tip assembly 122 may then be removed from the guide tube 120, such as by using a scissors, for example. The positioning and aligning device 130 is then removed from the surgical field, leaving only the guide tube 120 positioned across the heart chamber in the desired position for delivery of the mitral valve splint 10.

If necessary or desired, it is possible to reposition the guide tube 120. The positioning and aligning device 130 at this stage has the tip 122 from the prior needle delivery assembly 110 in the central dock 192. This tip 122 may be rotated out of position, bringing one of the auxiliary docks 192 into alignment with the slope 188 of the capture plate cover 184 as described hereinbefore. The positioning and aligning device 130 may then be repositioned on the heart H as described before, and a different needle delivery assembly 110 may then be delivered in a new position following the same steps described above.

Once the guide tube 120 is deemed in an appropriate position, the mitral valve splint 10 may be delivered in a manner similar to the method described in the U.S. application Ser. No. 09/680,435, now U.S. Pat. No. 6,723,038, filed Oct. 6, 2000, entitled METHODS AND DEVICES FOR IMPROVING MITRAL VALVE FUNCTION (hereinafter the '435 application), the entire disclosure of which is incorporated by reference. The tension member 12 is provided with the posterior (fixed) pad 16, or at least the block 42 of the releasable connection mechanism 40, connected thereto. The tension member 12 may include a leader section (not shown) that is advanced into the now accessible posterior (distal) end of the guide tube 120. Once the leader of the tension member 12 emerges from the anterior (proximal) end of the guide tube 120, the leader of the tension member 12 and the guide tube 120 are pulled proximally, placing the posterior anchor pad 16 in position on the epicardium. The anterior (adjustable) pad 14 is then positioned on the tension member 12. A measuring and tightening device such as that described in U.S. Pat. No. 6,260,552 to Mortier et al., the disclosure of which is incorporated herein by reference, may be used to adjust the spacing of the anterior and posterior pads 14, 16 to an optimum distance. Mitral valve function may be observed with appropriate diagnostic techniques such as transesophageal echocardiography (TEE) to assist in determining the appropriate distance between the anterior and posterior pads 14,16 and the appropriate tightness of the splint 10.

Once the splint 10 is appropriately tightened, the anterior pad 14 is secured to the tension member 12, similar to the method described in the '435 application, incorporated herein. At any time during delivery of the splint 10, the posterior pad 16 may be switched to a pad of a different shape or size, as described hereinbefore, by utilizing the releasable connection mechanism 40. Once the proper posterior pad 16 is in place and the desired mitral valve function is established and confirmed using an appropriate diagnostic method, the thoracotomy may be closed.

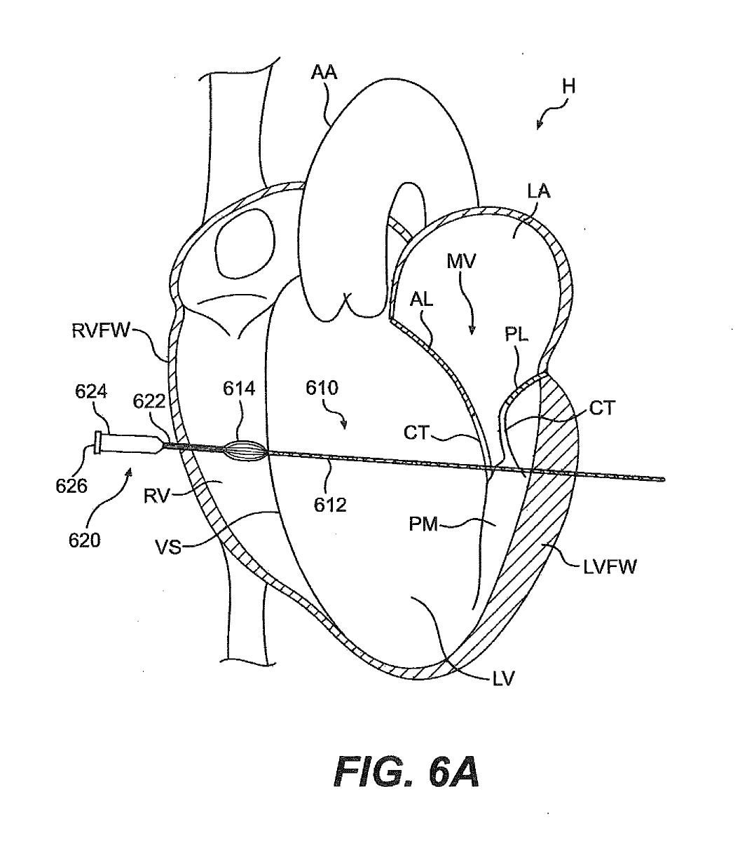

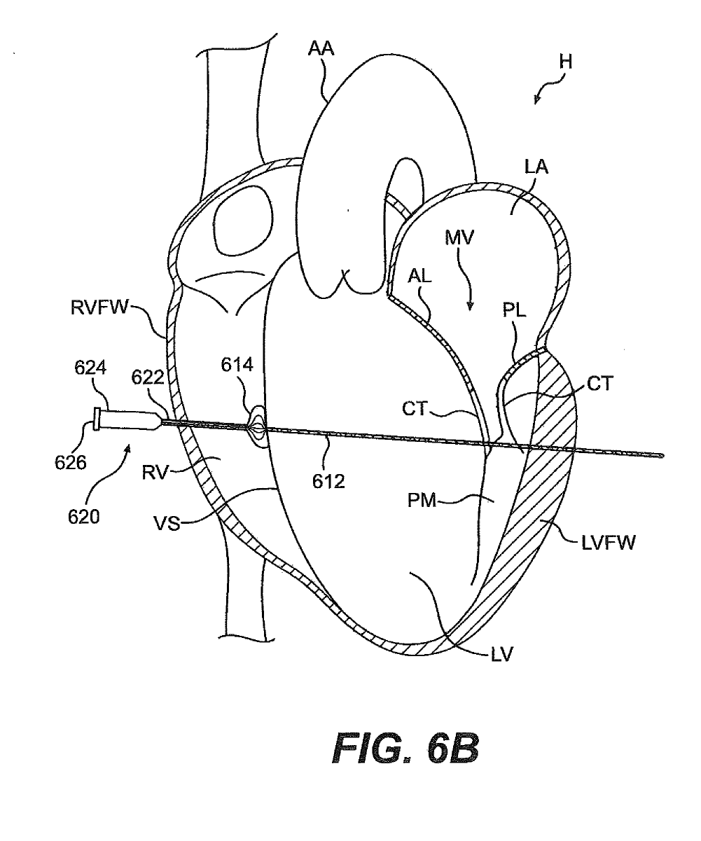

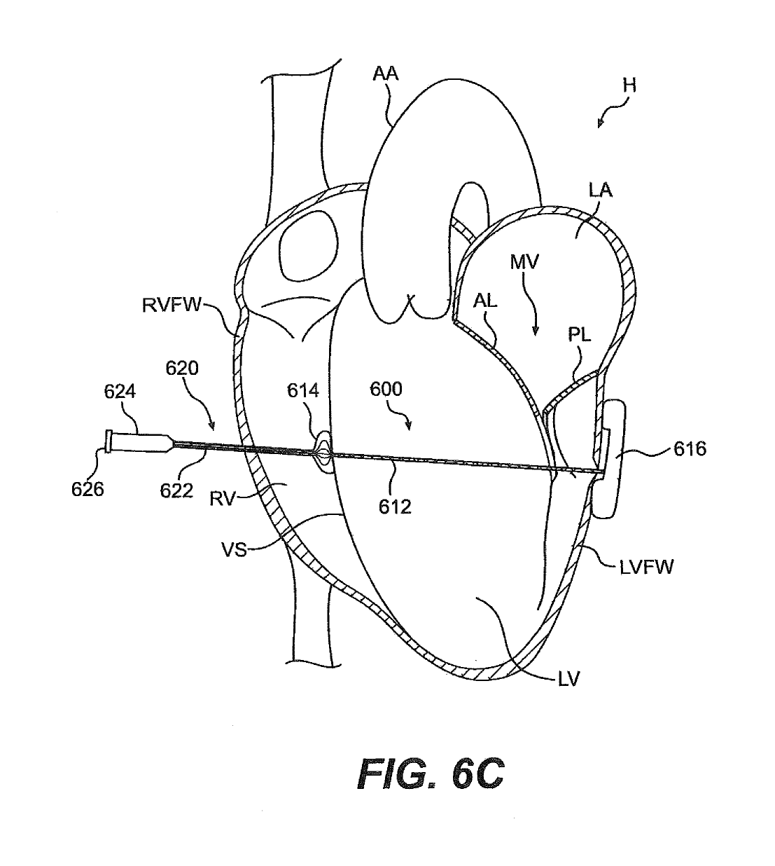

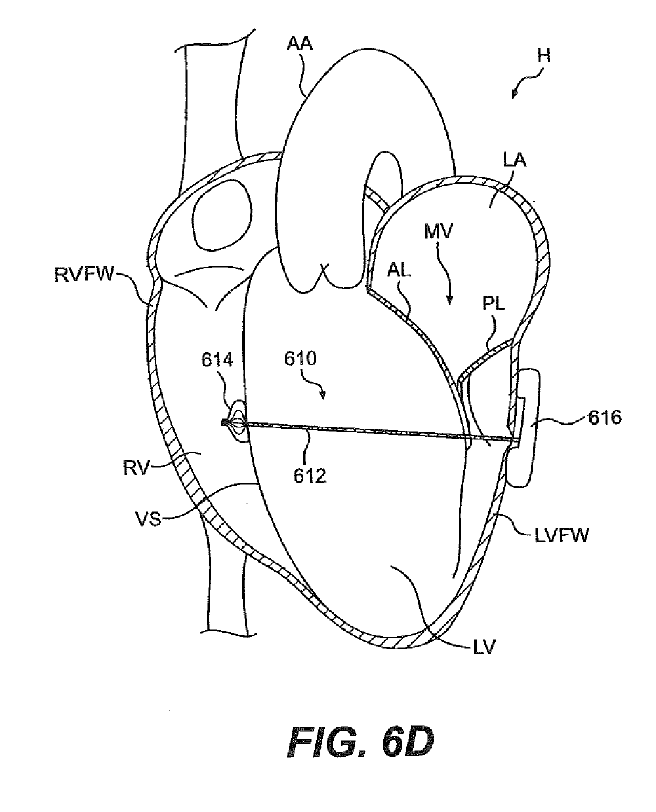

With reference to FIGS. 6A-6D, exemplary embodiments of a septal mitral valve splint 610, septal delivery system and septal delivery method are schematically illustrated, which may be similar to that described with reference to the epicardial mitral valve splint 10, except as apparent from the drawings and related discussion. As best seen in FIG. 6D, the septal mitral valve splint 610 generally includes a tension member 612, a septal anchor 614, and a posterior (epicardial) pad 616. Tension member 612 may be similar to tension member 12, and posterior pad 616 may be similar to posterior pad 16.

A general difference between the septal approach illustrated in FIGS. 6A-6D and the epicardial approach illustrated in FIGS. 1A-1C is that the anterior (epicardial) pad 14 has been replaced by a septal anchor 614 that may be located more superiorly, thus altering the force vector of the tension member 12. The septal approach may be better suited for certain types of mitral valve dysfunction than the epicardial approach. However, as with the epicardial approach, the septal approach causes local deformation of the annulus AN of the mitral valve MV and brings the posterior leaflet PL in better apposition to the anterior leaflet AL. In addition, one or both papillary muscles PM may be repositioned, further facilitating leaflet apposition and minimizing mitral valve regurgitation.

To facilitate delivery of the septal splint 610, a balloon-tipped probe 620 may be utilized. The probe 620 may include an elongate shaft 622 having a length sufficient to extend across the right ventricle RV to the ventricular septum VS as shown in FIG. 6A. A handle 624 having an inflation port 626 is connected to the proximal end of the shaft 622 and a balloon 614 is detachably connected to the distal end of the shaft 622. The shaft 622 may include an inflation lumen that defines a fluid path between the inflation port 626 and the interior of the balloon 614 to permit the balloon 614 to be selectively inflated and deflated by utilizing a syringe (not shown) or other suitable inflation device connected to the port 626. The balloon 614 may be formed of PET or other similar suitable material, and may be fixedly connected to the proximal end of the tension member 612. However, the shaft 622 may optionally include a tension member lumen to accommodate the tension member 612 therein. The tension member lumen may extend through the balloon 614 and all or a portion of the elongate shaft 622 and handle 624.

In use, a guide tube (not shown in FIGS. 6A-6D), similar to guide tube 120 discussed above, may be delivered across the right ventricle RV and left ventricle LV utilizing the delivery system 100 and related method described previously, but with a different orientation as shown in FIG. 6A. The tension member 612, with its proximal end fixedly connected to the balloon 614, may then be threaded through the guide tube from the anterior side to the posterior side, and the guide tube may be subsequently removed. The distal (posterior) end of the tension member 612 may be pulled posteriorly, to pull the probe 620 through the right ventricular free wall RVFW and right ventricle RV until the balloon 614 abuts the ventricular septum VS as shown in FIG. 6A.

A syringe (not shown) or other suitable inflation device may then be connected to the port 626 of the handle 624. The syringe may contain a curable inflation fluid such as, for example, a bone cement. The syringe may then be used to inflate the balloon 614 with the curable material as seen in FIG. 6B. The inflated balloon 614 may have a conical geometry, for example, that provides a larger surface area against the ventricular septum VS. The tension member 612 may be embedded in the curable material residing in the balloon 614 to provide a more effective bond therebetween. A posterior pad 616 may then be connected to the distal end of the tension member 612. After the material in the balloon 614 has cured, the posterior pad 616 may be adjusted on the tension member 612 to adequately tighten the splint 600 and force the leaflets AL, PL into full apposition, as shown in FIG. 6C. The balloon 614 may then be detached from the shaft 622 and the probe may be removed as shown in FIG. 6D.

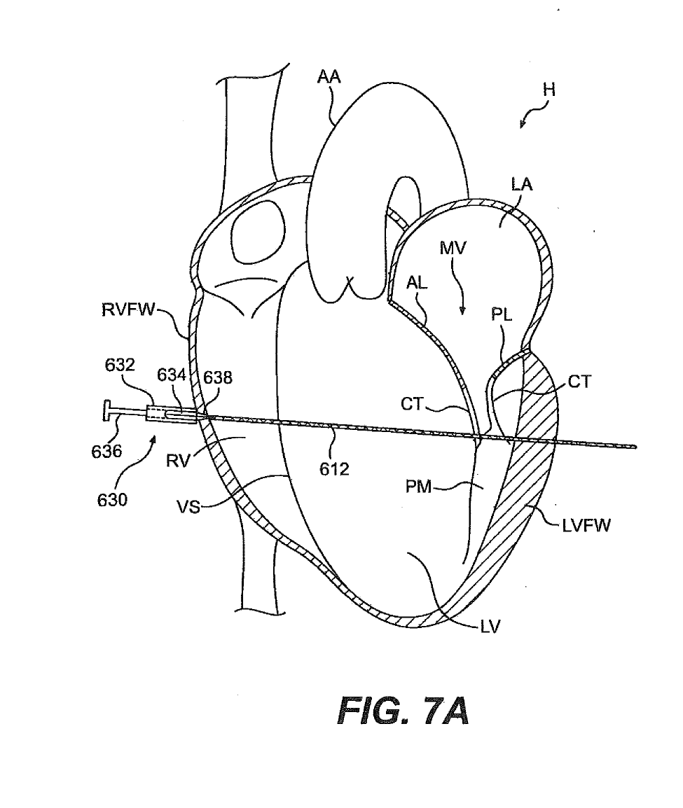

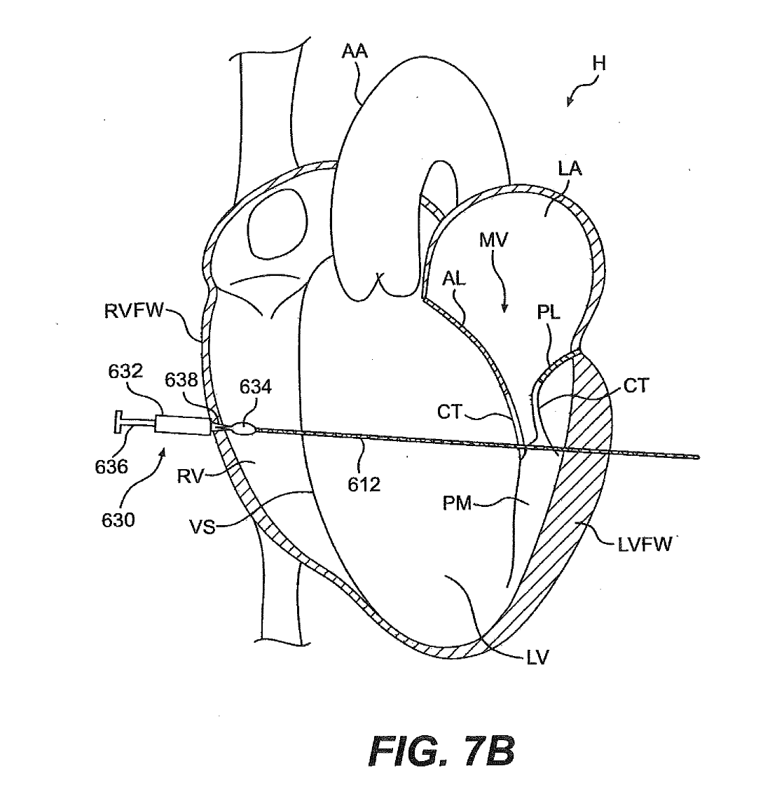

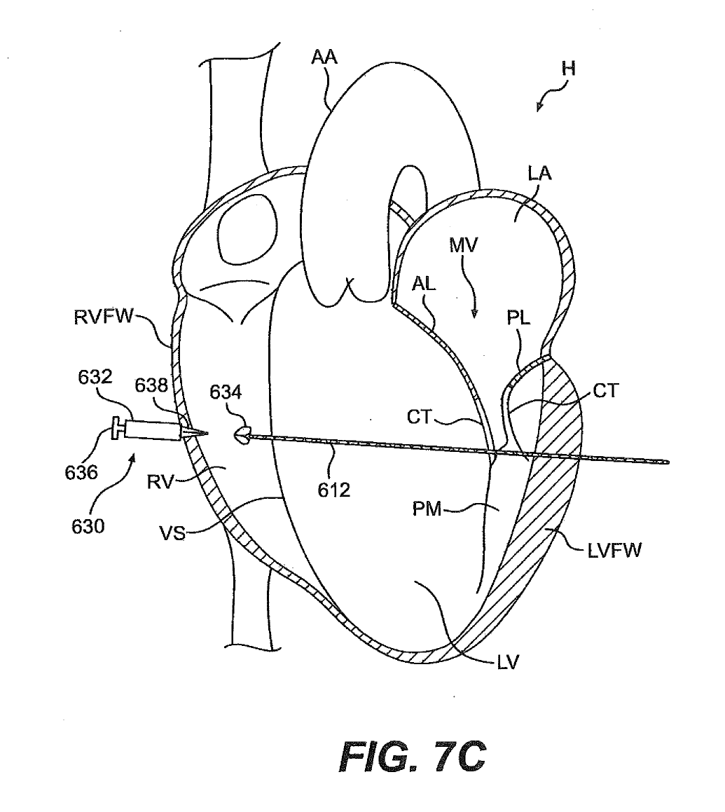

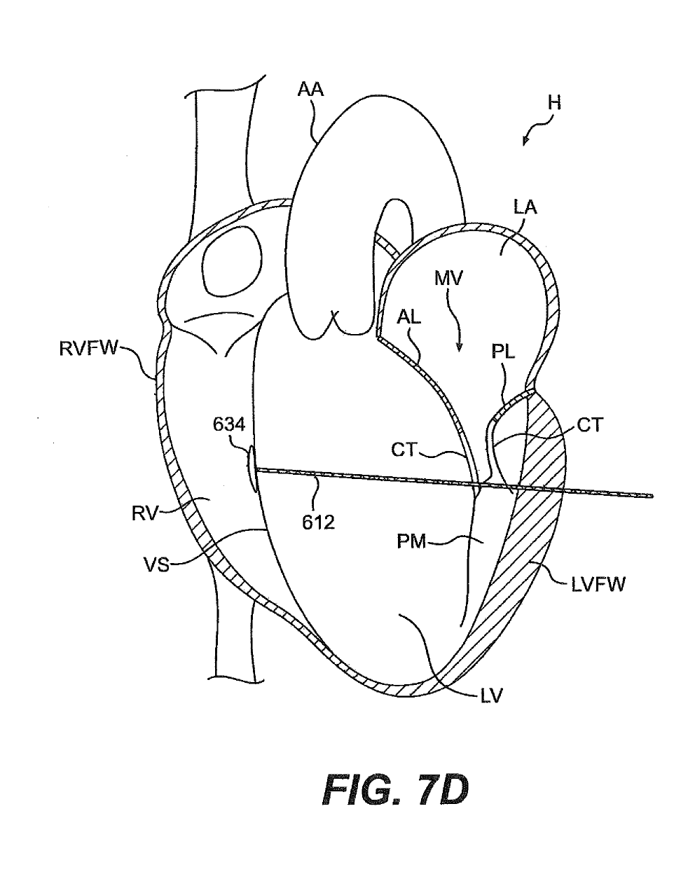

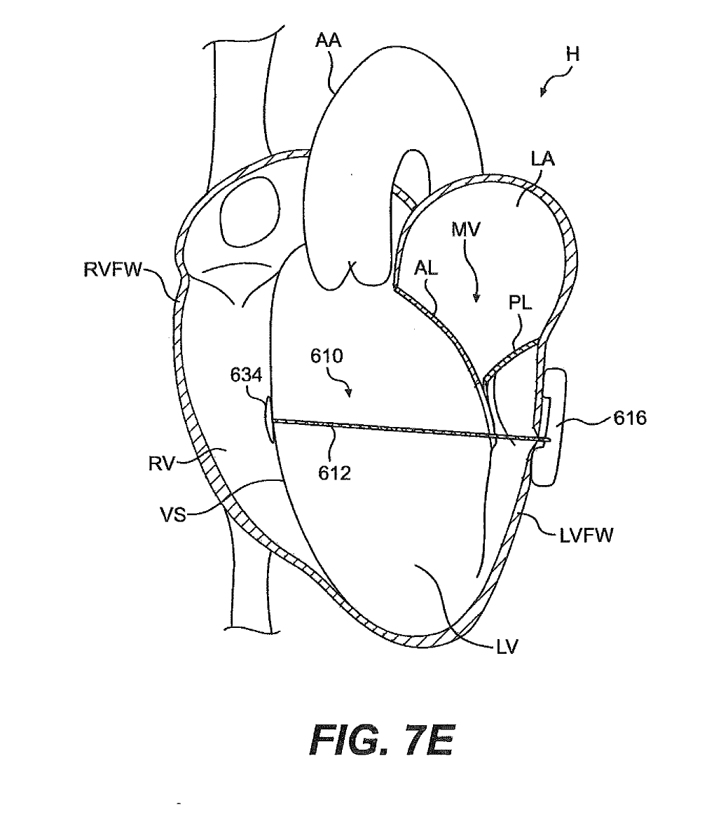

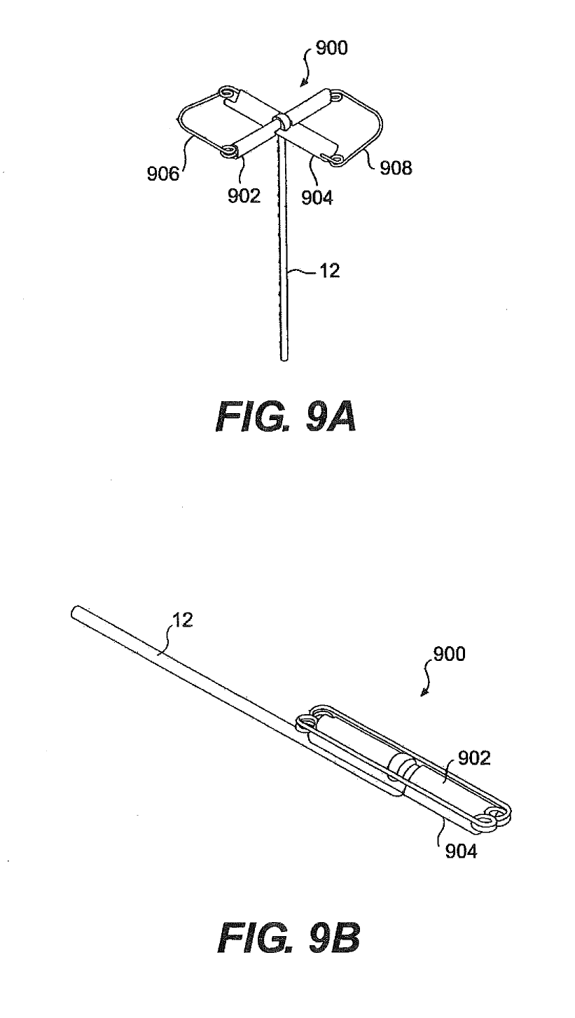

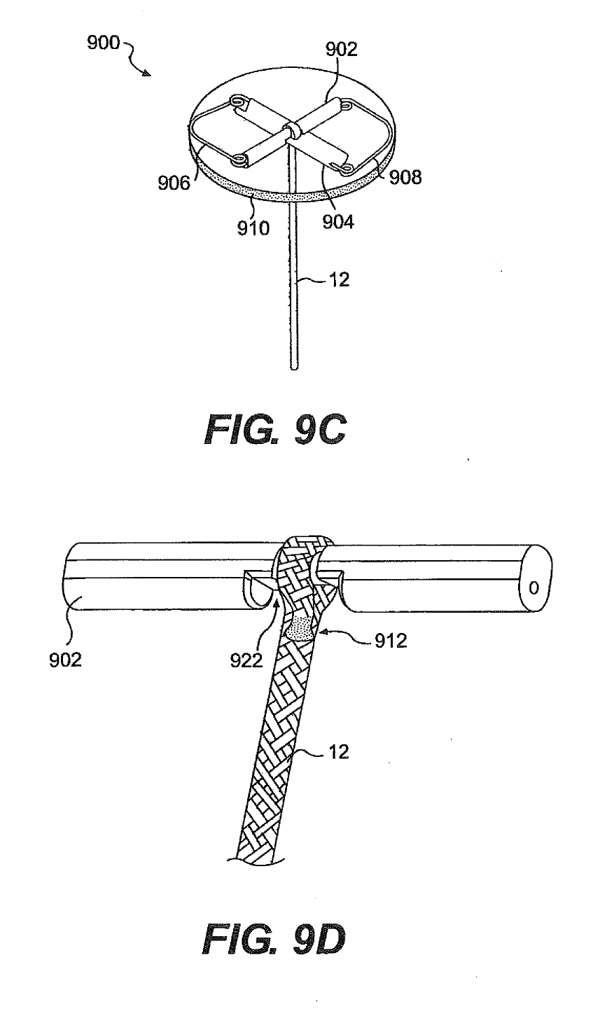

With reference to FIGS. 7A-7E, schematic illustrations of exemplary embodiments of an alternative septal pad 634 and delivery system are shown for the mitral valve splint 610 described with reference to FIGS. 6A-6D. The primary difference between the septal approach illustrated in FIGS. 7A-7E and the septal approach illustrated in FIGS. 6A-6D is that the septal balloon pad 614 has been replaced by a self expanding septal pad 634. Other aspects may remain the same or similar. As best seen in FIG. 7E, the septal mitral valve splint 610 generally includes a tension member 612, a septal anchor 634, and a posterior (epicardial) pad 616. The self expanding septal pad 634 may comprise any of the devices described with reference to FIGS. 9A-9D, for example, and may be fixedly connected to the proximal (anterior) end of the tension member 612.

To facilitate delivery of the self expanding septal pad 634, a delivery probe 630 may be utilized. Delivery probe 630 may include a barrel 632 defining a chamber therein which contains the self expanding septal pad 634 in a collapsed mode. A plunger 636 may extend into a proximal portion of the barrel 632. An expandable and sharpened tip 638 capable of penetrating the heart wall may be provided at the distal end of the barrel 632. Actuation of the plunger 636 in the distal direction with respect to the barrel 632 causes the self expanding septal pad 634 to be pushed into and through the tip 638, which may expand to accommodate the self expanding septal pad 634 therein.

In use, a guide tube similar to guide tube 120 (not shown) may be delivered across the right ventricle RV and left ventricle LV utilizing the delivery system 100 and related method described previously, but with a different orientation as compared to the orientation shown in FIG. 1A. The tension member 612, with its proximal end fixedly connected to the self expanding septal pad 634, may then be threaded through the guide tube from the anterior side to the posterior side, and the guide tube may be subsequently removed. The distal (posterior) end of the tension member 612 may be pulled posteriorly to pull the tip 638 of the probe 630 so that the tip 638 penetrates the right ventricular free wall RVFW as shown in FIG. 7A. The tension member 612 may continue to be pulled posteriorly until the self expanding septal pad 634 exits the tip 638 of the probe 630, as shown in FIG. 7B, enlarges to its expanded mode as shown in FIG. 7C, and abuts the ventricular septum VS as shown in FIG. 7D. A posterior (adjustable) pad 616 may then be connected to the distal end of the tension member 612 and adjusted to adequately tighten the splint and force the leaflets AL, PL into full apposition, as shown in FIG. 7E.

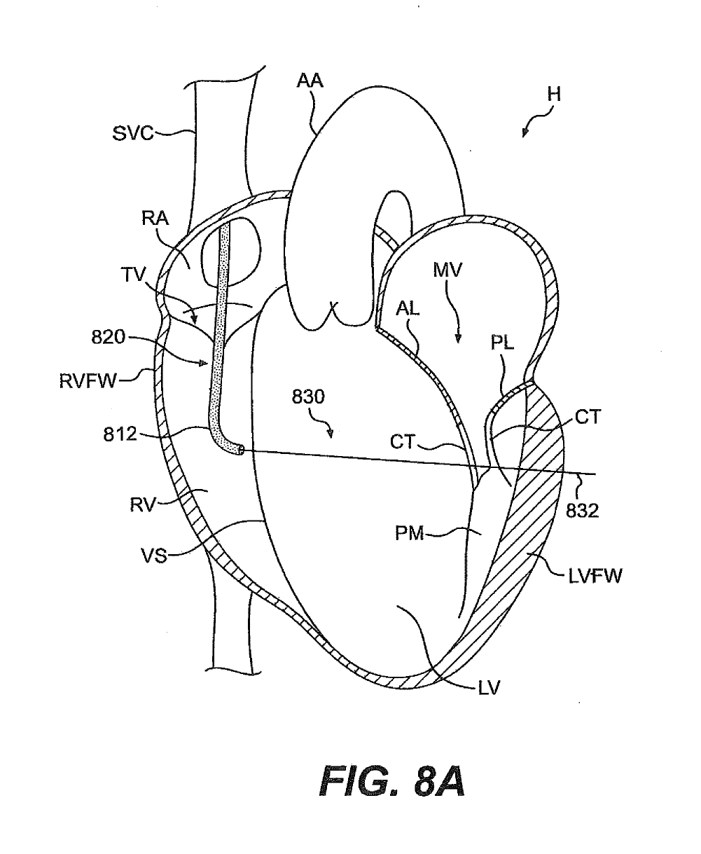

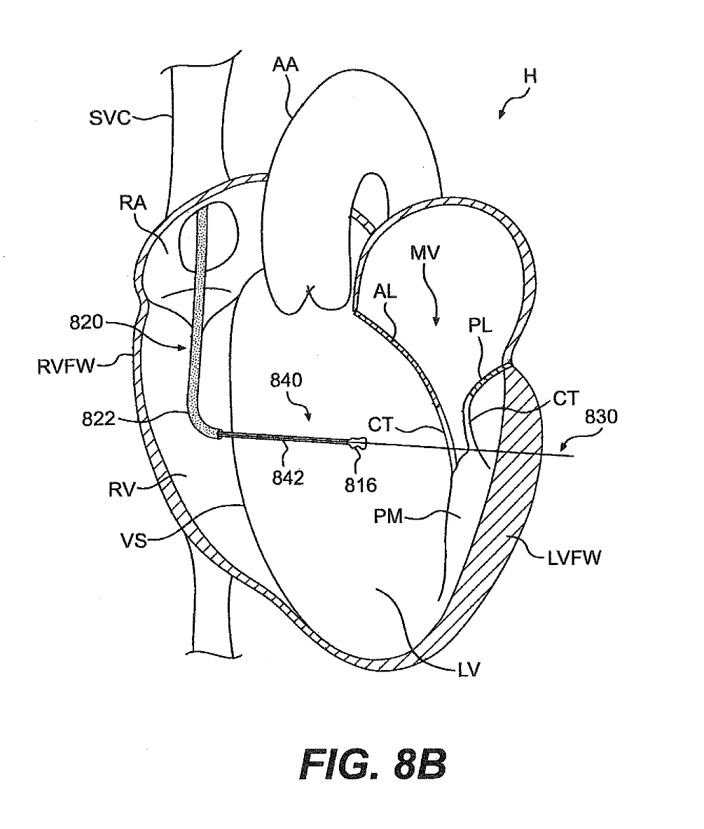

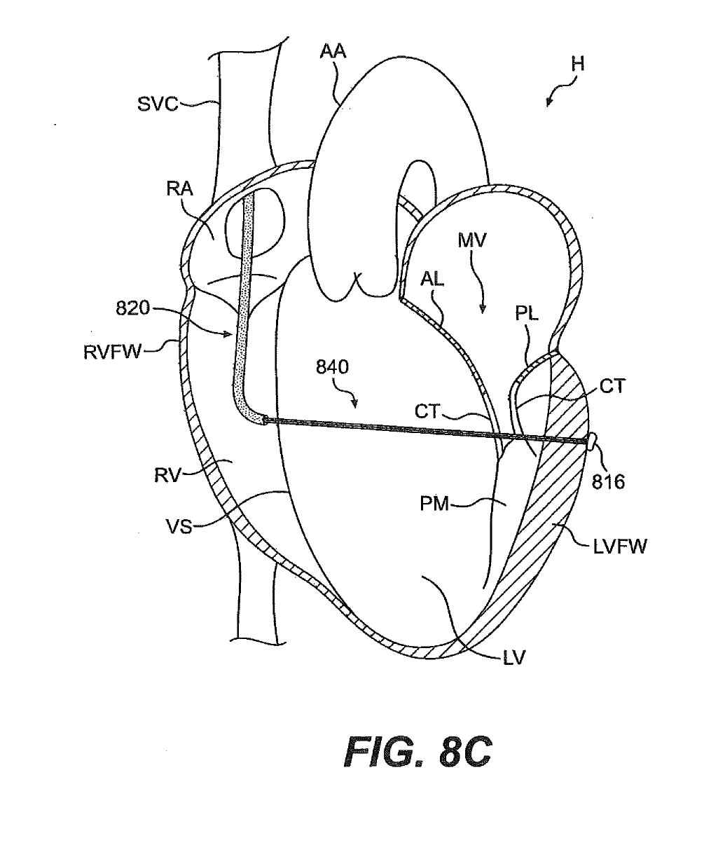

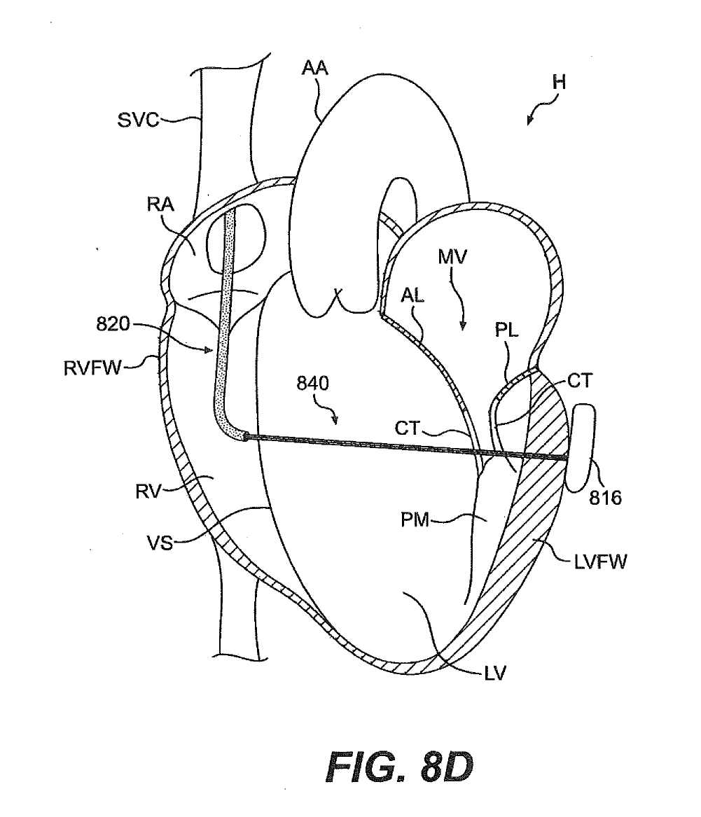

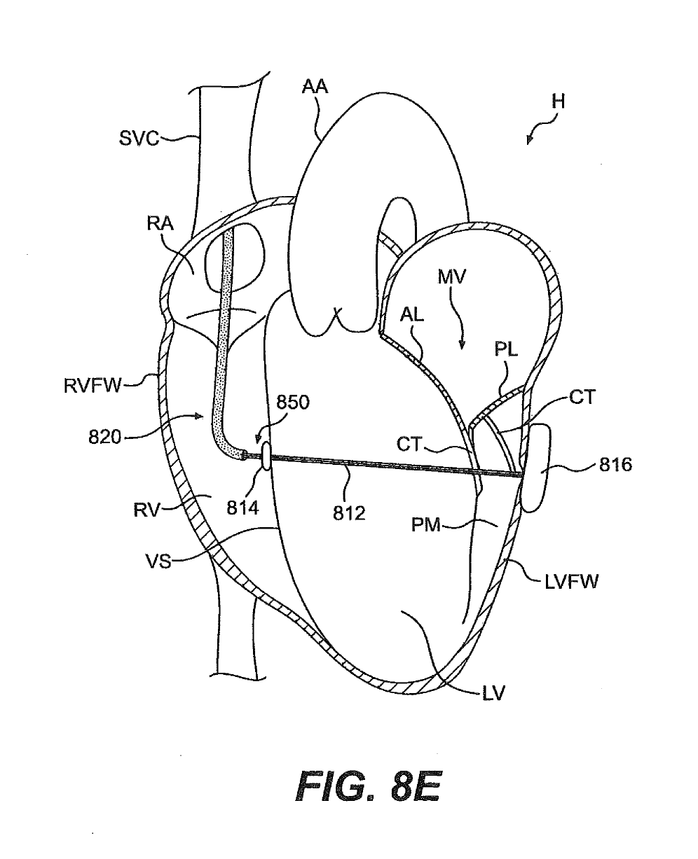

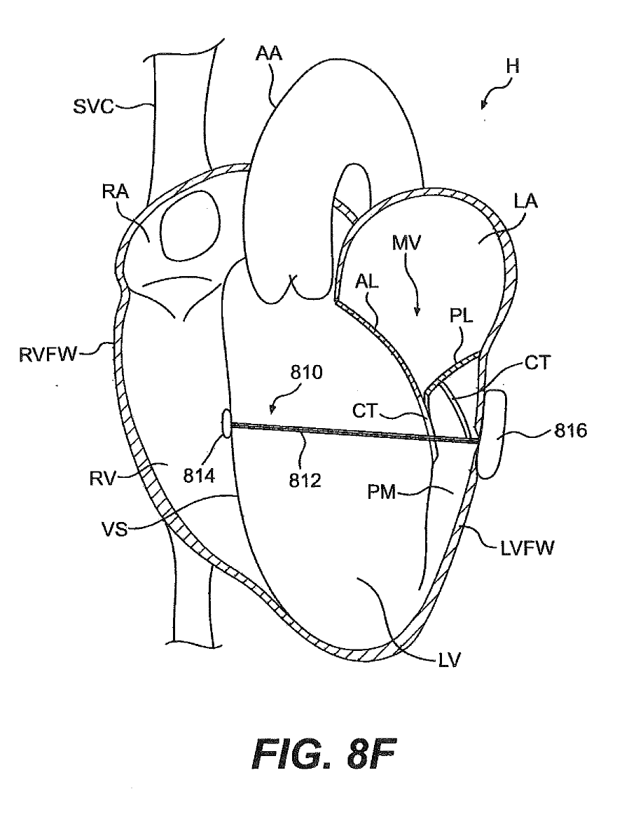

With reference to FIGS. 8A-8F, schematic illustrations of exemplary embodiments of yet another septal splint 640 and delivery method are shown. The septal approach illustrated in FIGS. 8A-8F is generally different than those described hereinbefore in that it is an endovascular approach, but other aspects may remain the same or similar to those described with reference to FIGS. 6A-6D. More details of an endovascular approach may be found in U.S. patent application Ser. No. 09/679,550, now U.S. Pat. No. 6,616,684, entitled ENDOVASCULAR SPLINTING DEVICES AND METHODS, the entire disclosure of which is incorporated herein by reference.

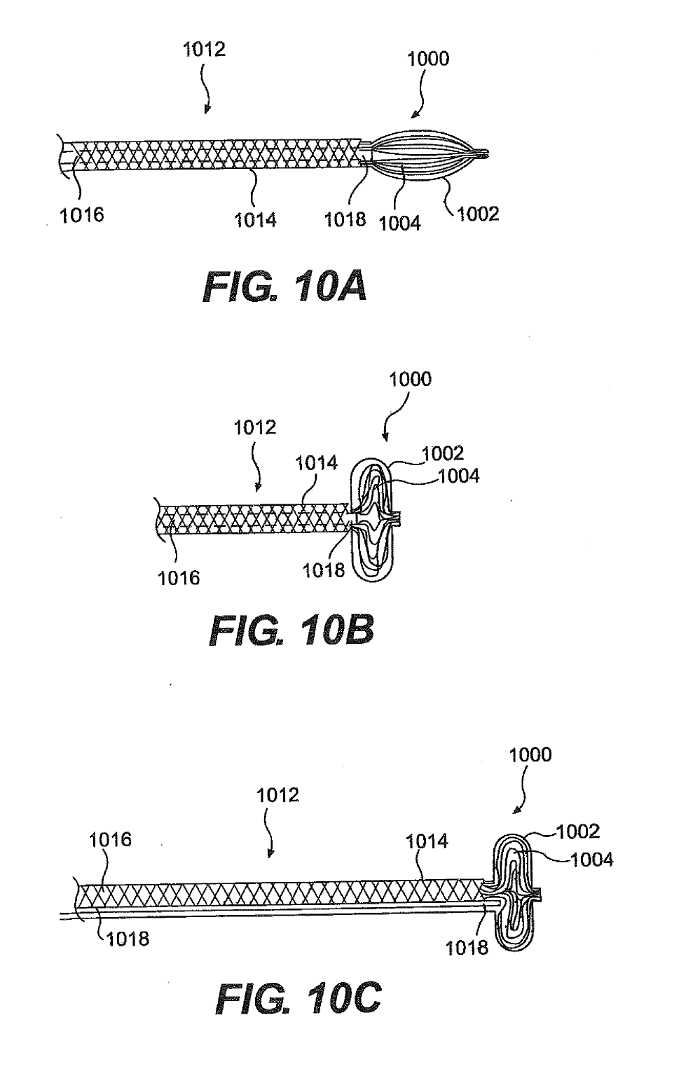

As best seen in FIG. 8F, the endovascular septal mitral valve splint 810 generally includes a tension member 812, a septal pad 814, and a posterior (epicardial) pad 816. The septal and epicardial pads 814, 816 may comprise, for example, any of the devices described with reference to FIGS. 10A-10C. Tension member 812 may be the same as or similar to tension member 12.

In use, a guide catheter 820 may be navigated through a patient's vascular system until the distal end thereof resides within the right ventricle RV. For example, the guide catheter 820 may be navigated from the peripheral veins in the arm to the superior vena cava SVC, through the right atrium RA, past the tricuspid valve TV, and into the right ventricle RV. The distal end of the guide catheter 820 includes a curved portion 822 to direct the distal end of the guide catheter 820 at the ventricular septum VS. Once the guide catheter 820 is in this position, a guide wire 830 may be inserted through the guide catheter 820. A tissue penetrating tip (e.g., sharpened tip) 832 of the guide wire 830 may pass through the ventricular septum VS, across the left ventricle LV, and through the left ventricular free wall LVFW as shown in FIG. 8A.

A balloon-tipped catheter 840 may then be passed over the guide wire 830 as shown in FIG. 8B. The balloon catheter 840 includes an elongate shaft 842 that extends through the guide catheter 820. A detachable balloon 816 may be connected to the distal end of the shaft 842, and may be formed of PET, for example. The elongate shaft 842 may include a guide wire lumen and an inflation lumen (not visible). The inflation lumen is in fluid communication with the balloon 816 and an inflation port (not visible) connected to a proximal end of the shaft 842. The guide wire lumen may extend through the balloon 816 and all or a portion of the shaft 842. The tension member 812 (not visible) is fixedly connected to the balloon 816 and extends proximally in the shaft 842 of the catheter.

The balloon catheter 840 may then be urged distally over the guide wire 830 until the balloon traverses the left ventricular free wall LVFW as shown in FIG. 8C, and the guide wire 830 may be removed. A syringe (not shown) or other inflation device may then be connected to the inflation port at the proximal end of the catheter 840. The syringe may contain a curable inflation fluid such as, for example, a bone cement. The syringe may then be used to inflate the balloon 816 with the curable material as seen in FIG. 8D. The balloon 816 may have an asymmetric inflated geometry, for example, that extends superiorly adjacent the annulus AN of the mitral valve MV, and that provides a large atraumatic surface area against the epicardial surface as seen in FIG. 8D. Alternatively, the balloon 816 may have a symmetric inflated geometry. Once cured, the catheter shaft 842 may be detached from the balloon 816, leaving the balloon 816 as the posterior epicardial pad and leaving the tension member 812 extending across the left ventricle as shown in FIG. 8E.

Using the tension member 812 as a substitute for the guide wire 830, another balloon-tipped catheter 850 may then be passed over the tension member 812. The balloon catheter 850 is similar to balloon catheter 840, except that balloon 814 may be secured to the tension member 812 upon curing. The second balloon catheter 850 may be urged distally until the balloon 814 engages the ventricular septum VS is inflated with a curable material. With the posterior balloon 816 in the desired location and the distal end of the tension member 812 fixed thereto, the tension member 812 may be pulled proximally while pushing on the second balloon catheter 850 to force the leaflets AL, PL into full apposition as shown in FIG. 8E. The balloon 814 of the second balloon catheter 850 is allowed to cure, thus securing the tension member 812 to the balloon 814, which then becomes the septal pad 814. The balloon 814 is detached from the remainder of the catheter 850. The tension member 812 may then be cut adjacent the proximal side of the septal pad 814, and the catheters 820, 850 may be removed, thus leaving splint 810 implanted as shown in FIG. 8F.

With reference to FIGS. 9A-9D, perspective views of a self expanding pad 900 and associated components are shown. The self expanding pad 900 may be used with the septal mitral valve splints of FIGS. 6-8, for example, as discussed above. The self expanding pad 900 is expandable between a collapsed delivery configuration as shown in FIG. 9B, and an expanded deployed configuration as shown in FIG. 9A. The small profile (diameter) of the self expanding pad 900 in the collapsed configuration permits the pad 900 to be delivered through a low-profile catheter or probe as described with reference to FIGS. 6-8, while the large profile of the self expanding pad 900 enables the pad to effectively and a traumatically engage the epicardium or septum, while resisting being pulled therethrough by the tension member 12.

Self expanding pad 900 includes a first arm 902 and a second arm 904 that pivot at their midpoints. The tension member 12 is fixedly connected to the first arm 902 and extends through a central hole in the second arm 904, thus pivotally connecting the two arms 902, 904. Two spring members 906, 908 are connected to the ends of the first and second arms 902, 904 as shown, to provide a biasing force on the arms 902, 904 rendering them self-expandable. The two spring members 906, 908 may be formed of spring tempered stainless steel, for example, or other suitable material. The first arm 902 and the second arm 904 may be formed of a stainless steel hypotube stock, for example, or other suitable material.

The first arm 902 may have a circular cross-section and the second arm 904 may be crimped to define a c-shaped or u-shaped cross-section. With this geometry, the first arm 902 rests in the second arm 904 (in the collapsed configuration) to create a toggle between the collapsed configuration and the expanded configuration. The first arm 902 defines a central recess 922 (visible in FIG. 9D) that is slightly wider than the width of the second arm 904 to accommodate and lock the second arm 904 in the expanded configuration.

As shown in FIG. 9C, the self expanding pad 900 may include a covering 910 formed of a velour woven polyester material, for example, available under the trade name Dacron™, or other similar suitable material such as, for example, expanded polytetrafluoroethylene (ePTFE). The covering 910 facilitates ingrowth of the heart wall tissue to secure the pad 900 to the epicardium or septum and thereby prevent long-term, motion-induced irritation thereto.

As shown in FIG. 9D, the tension member 12 may be connected to the first arm 902 by a tubular braid connection 912. In this exemplary embodiment, the inner cable of the tension member 12 may comprise a tubular braid, with one end of the tubular braid wrapped around the recess 922 of the first arm 902 and inserted into a hole at connection 912. When tensile forces are applied to the connection 912, the tubular braid constricts thereby locking down on the end inserted through the hole, similar to a Chinese finger lock.

With reference to FIGS. 10A-10C, perspective views of an expandable balloon pad 1000 and associated components are shown for use with the septal mitral valve splints of FIGS. 6-8, for example. The expandable balloon pad 1000 is connected to the distal end of a catheter shaft 1012, that may be detachable or that may serve as tension member 12. The expandable balloon pad 1000 includes an outer balloon 1002 formed of a thin polymer such as PET, for example. The distal end of the outer balloon 1002 is closed and sealed about the distal end of cable filaments 1004. The cable filaments 1004 may comprise the same or similar filaments forming the cable core of the tension member 12, for example. The filaments 1004 may extend proximally from the sealed distal end of the balloon 1002 and into the catheter shaft 1012.

The catheter shaft 1012 includes an outer tube 1014 to which the proximal end of the balloon 1002 is bonded and sealed. The catheter shaft 1012 also includes an inner tube 1018 disposed in the outer tube 1014 which defines an inflation lumen extending therethrough in fluid communication with the interior of the balloon 1002. The shaft 1012 may include a braid reinforcement 1016 carried in or under the outer tube 1014 to provide the same properties as the tension member 12. The braid reinforcement 1016 may comprise a continuation of the filaments 1004 extending from the balloon 1002. Alternatively, braid reinforcement 1016 may comprise a separate component and the proximal end of the filaments 1004 may be connected to the bond site between the balloon 1002 and outer tube 1014. If the braid reinforcement 1016 comprises a continuation of the filaments 1004 extending from the balloon 1002, the filaments 1004 forming the braid may extend coaxially around the inner tube 1018 as shown in FIGS. 10A and 10B, or extend adjacent the inner tube 1018 as shown in FIG. 10C.

A syringe (not shown) or other inflation device may be connected to the proximal end (not shown) of the shaft 1012 to communicate with the inflation lumen of the inner tube 1018. The syringe may contain a curable inflation fluid such as bone cement. The syringe may then be used to inflate the balloon 1002 with the curable material as seen in FIGS. 10B and 10C. The inflated balloon 1002 may have disc geometry, for example, that provides a larger surface area against the epicardium or septum. The filaments 1004 may be embedded in the curable material residing in the balloon 1002 to provide a more effective bond therebetween.

In yet another embodiment, a device for improving the function of a heart is provided. The device may include an elongate member for drawing at least two walls of the heart toward each other to reduce the radius or area of one or more heart chambers in at least one cross sectional plane. The elongate member may have an anchoring member disposed at opposite ends for engagement with the heart or chamber wall. Further, the elongate member may be configured to store contractile energy exerted by a heart chamber during, e.g., systole and release the stored energy during diastole to assist the heart chamber in expansion during, e.g., diastole. The elongate member may include a spring or spring like portion which compresses during systole to store energy, and releases the stored energy to help the elongate member to return to an uncompressed state, thereby assisting the chamber of the heart to expand during diastole.

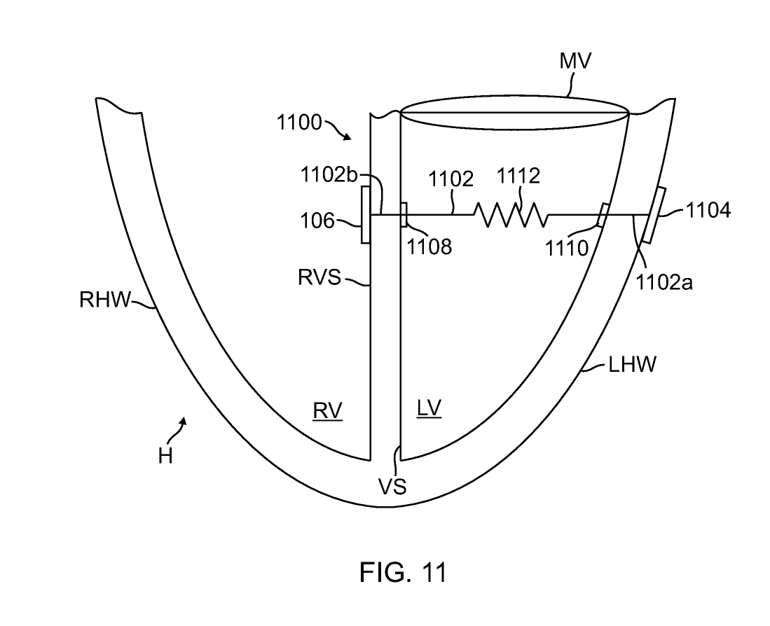

Turning now to FIG. 11, there is depicted an exemplary embodiment of a heart treatment device 1100. Device 1100 may include any of the features described in connection with the splint 10 of the aforementioned embodiments. In one embodiment, device 1100 may include an elongate member 1102. Elongate member 1102 may be substantially similar to tension member 12 in one or more ways. In some embodiments, portions of elongate member 1102 may be substantially rigid so as to be configured to exert a force on an inner surface of a heart wall, as will be discussed in greater detail below. Elongate member 1102 may include a first end 1102a and a second end 1102b. A first anchoring member, such as, for example, anchor pad 1104, may be disposed at first end 1102a. In addition, a second anchoring member, such as, for example, anchor pad 1106 may be disposed at second end 1102b. Anchor pads 1104, 1106 may include any suitable configuration known to those of ordinary skill in the art. For example, anchor pads 1104, 1106 may be self-expandable. In addition, anchor pads 1104, 1106 may include structures (not shown) disposed therein to increase the rigidity and/or aid in the expansion of anchor pads 1104, 1106. In some embodiments, anchor pads 1104, 1106 may be substantially similar to anchor pads 14, 16 in one or more ways.

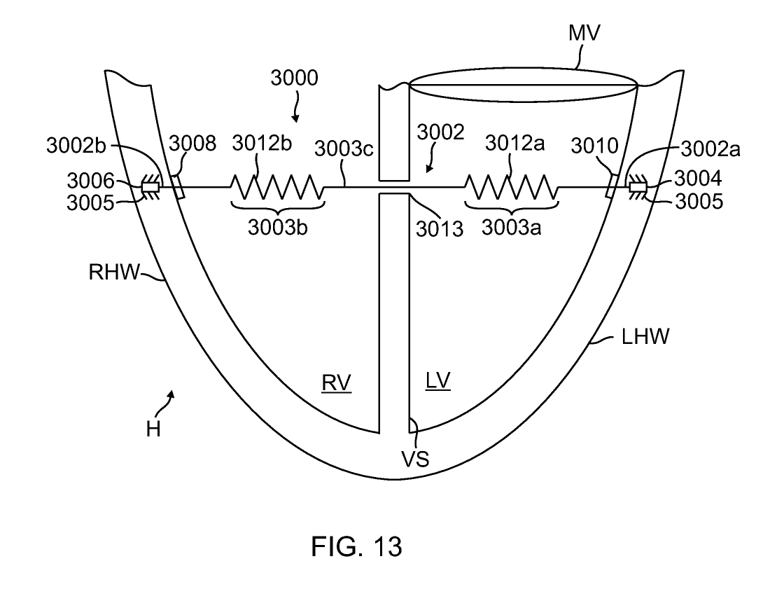

As shown in FIG. 11, anchor pads 1104, 1106 may be configured to secure device 1100 to a portion of heart H, so that elongate member 1102 may traverse the left ventricle LV of heart H. Although the embodiment depicted in FIG. 11 traverses the left ventricle LV, those of ordinary skill in the art will readily recognize that device 1100 may be implanted so as to traverse the right ventricle RV, or both ventricles, as discussed in connection with FIG. 13.

Further, a plurality of devices 1100 may be implanted on heart H. In one such embodiment, a first device 1100 may be implanted across the left ventricle LV, and a second device 1100 may be implanted across the right ventricle RV. In another embodiment, one or more devices 1100 may be implanted across a single heart chamber. Further, the use of device 1100 may not be limited to a heart's ventricles. Indeed, device 1100 may be positioned to traverse one or both of a heart's atria.

In the embodiment depicted in FIG. 11, anchor pad 1104 may be seated on the epicardium of the left ventricular heart wall LHW. In addition, anchor pad 1106 may be seated on the right ventricular surface RVS of the ventricular septum VS. Further, as alluded to above, anchor pads 1104, 1106 may include any suitable covering or coating known in the art. In some embodiments, such a covering or coating may be made of velour woven polyester material, for example, available under the trade name Dacron™, or other similar suitable material such as, e.g., expanded polytetrafluoroethylene (ePTFE). The covering or coating may be configured to facilitate ingrowth of the heart wall tissue to secure anchor pads 1104, 1106 to their respective surfaces and walls, and thereby prevent long-term, motion-induced irritation thereto.

In some embodiments, elongate member 1102 may be constructed from suitable biocompatible materials having elastic properties so that elongate member 1102 may have the elastic characteristics. Such materials may include, but are not limited to, nickel-titanium alloys, nickel-cobalt alloys, other cobalt alloys, thermoset plastics, thermoplastics, stainless steel, suitable shape-memory materials, suitable super-elastic materials, or combinations thereof, and the like. In other embodiments, elongate member 1102 may include a spring or spring-like portion 1112. Portion 1112 may include any suitable configuration known in the art. For example, portion 1112 may include a helical extension spring or a coil spring.

In all instances, however, elongate member 1102 may be configured to act substantially as a spring when implanted on a patient's heart H. For example, elongate member 1102 may be configured to compress and elongate as heart H naturally beats during a cardiac cycle. Generally, elongate member 1102 may be configured to compress as portions of heart H exert compressive forces on device 1100, such as, for example, during systole. During such compression, portion 1112 of elongate member 1102 may be configured to store energy (e.g., potential energy), and release the stored energy to help elongate member 1102 return to an uncompressed state when the compressive forces exerted by the heart are removed, such as, for example, during diastole. As a consequence of returning to its uncompressed configuration, elongate member 1102 may serve to assist associated portions of heart H expand during, for example, diastole.

In the specific embodiment of FIG. 11, elongate member 1102 may be compressed as the left ventricle LV contracts during systole. As the left ventricular heart wall LHW contracts to reduce the left venticle's LV volume, the left ventricular heart wall LHW will exert a compressive force on elongate member 1102, causing it to compress lengthwise. During the compression of elongate member 1102, portion 1112 may store potential energy. When the left ventricle LV expands during diastole, the compressive forces acting on elongate member 1102 during systole are reduced. Thus, the potential energy stored in portion 1112 may be released, causing elongate member 1102 to return to an uncompressed configuration. As elongate member 1102 returns to its uncompressed configuration, device 1100, as a result of being secured to the ventricular septum VS and the left ventricular heart wall LHW, may exert an outward force on the ventricular septum VS and the left ventricular heart wall LHW, thereby assisting the left ventricle to expand during diastole.

Although the embodiment depicted in FIG. 11 illustrates that spring or spring-like portion 1112 constitutes a central region of elongate member 1102, those of ordinary skill in the art will readily recognize that portion 1112 may include any suitable region of elongate member 1002. For example, in some embodiments, portion 1112 may constitute the entire length of elongate member 1002.

With continued reference to FIG. 11, device 1100 may further include force dispersion components 1108, 1110. Components 1108, 1110 may be configured to disperse the forces exerted on a heart wall (e.g., the left ventricular heart wall LHW) by elongate member 1102 as elongate member 1102 returns to an uncompressed configuration. Components 1108, 1110 may include any suitable configuration known in the art. In some embodiments, components 1108, 1110 may be similar to anchor pads 1104, 1106 in one or more ways. In other embodiments, components 1108, 1110 may include a configuration similar to a grommet or an eyelet. In further embodiments, components 1108, 1110 may be self-expandable. It is contemplated that components 1108, 1110 may be fixedly secured to elongate member 1102 by any suitable technique known in the art so that they are prevented from moving relative to elongate member 1102 as elongate member 1102 applies a force to an inner surface of a heart wall (e.g., the left ventricular heart wall LHW).

The elasticity or stiffness of portion 1112 may be configured so that portion 1112 may compress when subjected to contractile forces of the left ventricle LV. Similarly, the elasticity or stiffness of portion 1112 may be configured so that portion 1112 may cause elongate member 1102 to exert expansive forces on the associated heart walls. The elasticity or stiffness of portion 1112 may be determined using Hooke's Law (F=kx, where F=force, k=spring constant (i.e., stiffness), and x=displacement).

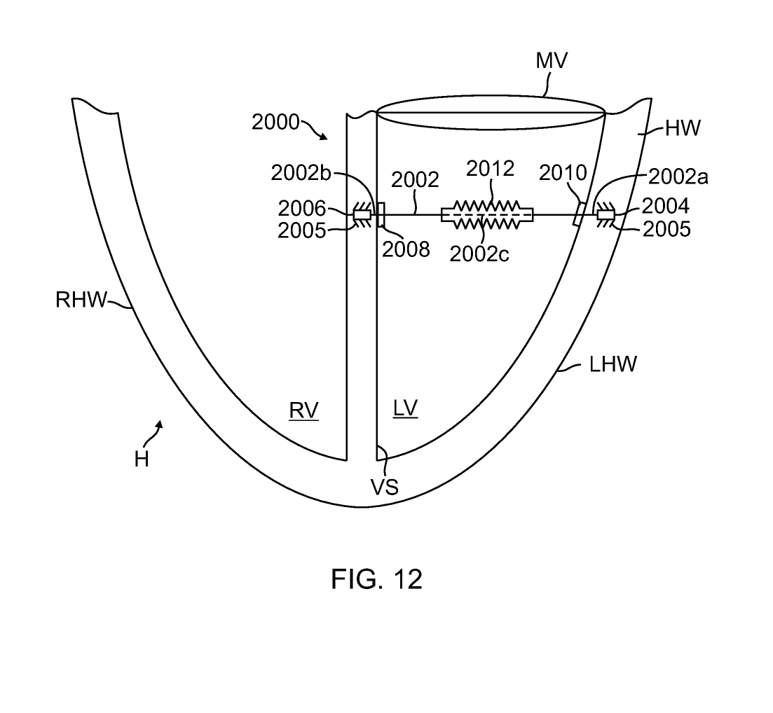

FIG. 12 depicts another exemplary embodiment of a heart treatment device 2000. Device 2000 may be substantially similar to device 1100 in one or more ways. As shown in FIG. 12, device 2000 includes an elongate member 2002 having ends 2002a, 2002b. Elongate member 2002 may be substantially similar to elongate member 1102 in one or more ways. Device 2000 further includes anchoring members 2004, 2006 disposed at ends 2002a, 2002b, respectively. Anchoring members 2004, 2006 may be configured for intra-myocardial retention and may include any suitable configuration known in the art. Stated differently, anchoring members 2004, 2006 may be configured to be disposed within a portion of the left ventricular heart wall LHW and/or the ventricular septum VS. In addition, anchoring members 2004, 2006 may be substantially similar to anchor pads 14, 16, 1104, 1106 described above. Each of anchoring members 2004, 2006 may include one or more anchoring elements 2005. Anchoring elements 2005 may include any suitable configuration known in the art. In some embodiments, anchoring elements 2005 may include tines projecting away from the body of anchoring members 2004, 2006. Anchoring elements 2005 may be oriented in such a manner so as to permit insertion of anchoring members 2004, 2006 into heart tissue and to resist removal of anchoring members 2004, 2006 from the heart tissue once inserted. Although the depicted embodiments illustrate three pairs of anchoring elements 2005 on each of anchoring members 2004, 2006, any suitable number of anchoring elements 2005 may be provided on anchoring members 2004, 2006. Further, anchoring members 2004, 2006 may be provided with differing numbers of anchoring elements 2005.

With continued reference to FIG. 12, device 2000 may also include a spring or spring-like portion 2012. Portion 2012 may be substantially similar to portion 1012 in functionality. Portion 2012, however, may be configured to be disposed over a region of elongate member 2002, such as, for example, region 2002c shown in phantom lines. Portion 2012 may include any suitable configuration known in the art. For example, portion 2012 may include a spring-like sleeve having a central longitudinal lumen within which region 2002c may be disposed. In addition, portion 2012 may be fixedly secured to region 2012c so that portion 2012 may compress as elongate member 2012 compresses. Device 2000 may also include force dispersion components 2008, 2010 that are similar to components 1108, 1110.

FIG. 13 depicts a further exemplary embodiment of a heart treatment device 3000. Device 3000 may be substantially similar to device 1100, 2000 in one or more ways. As shown in FIG. 13, device 3000 includes an elongate member 3000 having ends 3002a, 3002b. Elongate member 3000 may be substantially similar to elongate members 1102, 2002 in one or more ways. Device 3000 may further include anchoring members 3004, 3006 disposed at ends 3002a, 3002b, respectively. Anchoring members 3004, 3006 may include anchoring elements 3005, and may be substantially similar to anchoring members 2004, 2006 in one or more ways.

Device 3000 may be configured to traverse both the left ventricle LV and right ventricle RV, as shown in FIG. 13. Stated differently, anchoring member 3004 may be disposed within the left ventricular heart wall LHW. Elongate member 3002 may extend from anchoring member 3004, through an opening 3013 in the ventricular septum VS, and to the right ventricular wall RHW, within which anchoring member 3006 may be disposed.

Elongate member 3002 may include a left ventricular portion 3003a, a right ventricular portion 3003b, and a septal portion 3003c. Each of the left ventricular portion 3003a and right ventricular portion 3003b may include a spring or spring-like portion 3012a and 3012b. Portions 3012a and 3012b may be substantially similar to portion 1112 in functionality. In addition, each of portions 3012a and 3012b may be substantially similar in structure to portion 1112 in one or more ways. Device 3000 may also include force dispersion components 3008, 3010 that are similar to components 1108, 1110. Further, septal portion 3003c may include a section of elongate member 3002 having a reduced cross-section relative to, e.g., portions 3012a, 3012b, so as to minimize the size of opening 3013 in the ventricular septum VS. Furthermore, septal portion 3003c may be provided with a coating or covering configured to reduce irritation to the ventricular septum VS when portion 3003c moves relative to the ventricular septum VS. Although the embodiment of FIG. 13 depicts elongate member 3002 as having two spring or spring-like portions 3012a and 3012b, those of ordinary skill in the art will readily recognize that elongate member 3002 may include a greater or lesser number of spring or spring-like portions. Devices 1100, 2000, 3000 may be provided with one or more visualization features (not shown) configured to assist in visualizing devices 1100, 2000, 3000 from outside of the patient's body. Such features may include radiopaque material and/or sonoreflective markers configured to facilitate fluoroscopic visualization during implantation procedures.

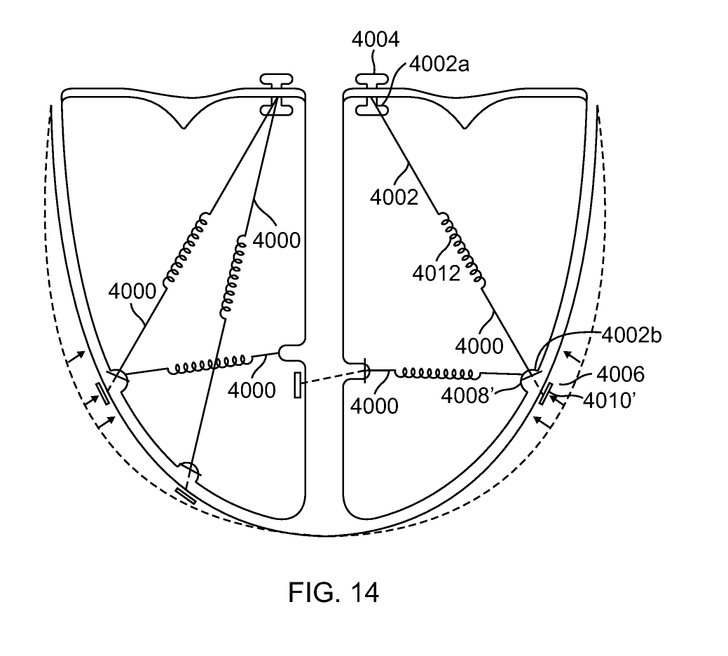

FIG. 14 depicts another exemplary embodiment of a heart treatment method where a plurality of heart treatment devices 4000 is shown. Device 4000 may be substantially similar to device 1100 in one or more ways. As shown in FIG. 14, device 4000 includes an elongate member 4002 having ends 4002a, 4002b. Elongate member 4002 may be substantially similar to elongate member 1102 in one or more ways. Device 4000 further includes anchoring members 4004, 4006 disposed at ends 4002a, 4002b, respectively. Anchoring members 4004, 4006 may be configured for intra-myocardial retention and may include any suitable configuration known in the art. Stated differently, anchoring members 4004, 4006 may be configured to be disposed within a portion of the left ventricular heart wall LHW and/or the ventricular septum VS. In addition, anchoring members 4004, 4006 may be substantially similar to anchor pads 14, 16, 1104, 1106 described above. The spring tension of the device 4000 enables lifting up of the dilated left ventricle wall. For example, the LV wall adjacent the posterior papillary muscle may be lifted up and in. This is achieved by pulling up on the tension member 4002 anchored at the trigon region. In one advantage, reshaping of the left ventricle may provide improved coaption of the mitral valve leaflets for eliminating or minimizing mitral insufficiency. It also provides a tension support for the weakened and diseased left ventricle wall to prevent further dilation.

In one embodiment, the anchoring members 4004, 4006 may be dumbbell shaped expandable anchoring member, with a distal end 4080′ and proximal end 4010′. The proximal end 4010′ rests on the epicardium, whereas the distal end 4008′ acts as a force dispersion member, similar to the pads 1108, 1110, 2008, 2010, 3008 and 3010.

With continued reference to FIG. 14, device 4000 may also include a spring or spring-like portion 4012. Portion 4012 may be substantially similar to portion 1012 in functionality. Portion 4012 may include any suitable configuration known in the art. A plurality of devices 4000 may be used extending through the right and the left ventricle of the heart. In one embodiment, the spring portion 4012 of the device 4000 exerts diagonal spring tension in a heart chamber. More specifically, the diagonal tension exerted by the spring element 4012 lifts up the dilating left ventricle wall. By lifting the left ventricle wall and anchoring the device 4000 on fibrous trigon region of the heart between the aortic valve and the mitral valve, the left ventricle size may be decreased. As discussed above, the trigon to left ventricle tether 4002, as shown in FIG. 14, enables supporting and reshaping the left ventricle.

The devices described in connection with FIGS. 11-14 may be utilized to improve heart function in conjunction with one or more additional devices/implants. For example, a plurality of devices 1000, 2000, 3000, 4000 may be utilized on a single heart H. In addition, devices 1100, 2000, 3000, 4000 may be utilized with any other device or method of therapy known in the art. For example, device 1100, 2000, 3000, 4000 may be used in conjunction with annuloplasty rings, external reconfiguration devices (e.g., bands or wraps), and/or coronary sinus implants.

Device 1100, 2000, 3000 may be utilized to treat a patient's heart H and/or otherwise improve the function of heart H. The following exemplary method will be described relative to device 1100, however, those of ordinary skill in the art will readily recognize that a similar method may be practiced with device 2000, 3000. In one exemplary embodiment, device 1100 may be delivered to a patient's heart H via a catheter (not shown) in substantially the same method as described in conjunction with FIGS. 8A-8F discussed above. In particular, the catheter may be navigated through a patient's vascular system until the distal end thereof resides within the right ventricle RV. For example, the catheter may be navigated from the peripheral veins in the arm to the superior vena cava SVC, through the right atrium, past the tricuspid valve TV, and into the right ventricle RV. Once the catheter is in this position, a guide wire, needle, or stylet (not shown) may be inserted through the catheter. A tissue penetrating tip (e.g., sharpened tip) of the guidewire, needle, or stylet may pass through the ventricular septum VS, across the left ventricle LV, and into or through the left ventricular heart wall LHW. Next, an anchor (e.g., anchor pad 1104) may be deployed into or through the left ventricular heart wall LHW for securing device 1100 to heart H. An elongate member 1102 may be then deployed from anchor pad 1104 across the left ventricle LV to either the ventricular septum VS or across the right ventricle RV to the right ventricular wall RHW, as determined by a clinician. Prior to finally anchoring elongate member 1102 to either the ventricular septum VS or right ventricular wall RHW, force dispersion components (e.g., 1108, 1110) may be deployed and positioned against inner surfaces of the left ventricular heart wall LHW and the ventricular septum VS or the right ventricular wall RHW. Then, anchor pad 1106 may be deployed to finally secure elongate member 1102 to heart H.

Once positioned, device 1100 may act as a spring to store potential energy when elongate member 1102 is compressed by the heart H during, e.g., systole. When the heart H expands during, e.g., diastole, and the compressive forces applied during systole are no longer present on elongate member 1102, elongate member 1102 may return to its uncompressed length by releasing the stored potential energy. In doing so, elongate member 1102 may apply outward forces to the heart walls via force dispersion components 1108, 1110 to assist the heart in expanding during diastole.

The methods disclosed herein may be performed by any suitable surgical technique known in the art, including, but not limited to, open surgery, minimally invasive or non-invasive surgery, endoscopically, percutaneously, and/or any combination thereof. In one embodiment, it is contemplated that the devices disclosed herein may be implanted within a patient via, for example, a minimally invasive surgical technique known as a thoractomy, such as, for example, an eight (8) centimeter thoractomy. In other embodiments, the embodiments described herein may be implanted within a patient's heart via a transapical procedure. In further embodiments, the depicted embodiments may be delivered via catheter-based techniques, including those techniques previously discussed herein. In addition, the methods described herein may be performed with or without the aid of cardiopulmonary bypass, as desired. For example, in one embodiment, the devices disclosed herein may be implanted and/or adjusted while heart function has been temporarily ceased and the patient is dependent upon cardiopulmonary bypass (i.e., on-pump). In another embodiment, however, the disclosed devices may be implanted and/or adjusted in accordance with the present disclosure without ceasing heart function (i.e., off-pump).

Those skilled in the art will recognize that the present disclosure may be manifested in a variety of forms other than the specific embodiments described and contemplated herein. Accordingly, departures in form and detail may be made and the present disclosure is intended to cover modifications and variations.

The above description presents the best mode contemplated for carrying out the present method and device for heart treatment, in such full, clear, concise, and exact terms as to enable any person skilled in the art to which they pertain to make this device and use these methods. This device and these methods are, however, susceptible to modifications and alternate constructions from those discussed above that are fully equivalent. Consequently, this system and these methods are not limited to the particular embodiments disclosed. On the contrary, this device and these methods cover all modifications and alternate constructions coming within the spirit and scope of the system and methods as generally expressed by the following claims, which particularly point out and distinctly claim the subject matter of the system and methods.

What is claimed is:

1. A method for improving the function of a heart, the method comprising:
providing a plurality of anchoring members;
providing an elongate member and a release mechanism connected to the elongate member, the release mechanism being configured to releasably engage with each of the plurality of anchoring members; the elongate member being configured to store energy exerted by a heart chamber during systole, and release the stored energy during diastole to assist the heart chamber to return to an uncompressed state;
selecting one of the plurality of anchoring members;
positioning the elongate member transverse a chamber of the heart; and
engaging the release mechanism with the selected anchoring member so as to releasably attach the elongate member to the selected anchoring member.

2. The method of claim 1, wherein providing an elongate member comprises providing the elongate member with a substantially rigid distal end, a substantially rigid proximal end and a substantially elastic portion disposed between the distal and the proximal end, where the substantially elastic portion is configured to compress during systole to store energy, and release the stored energy to help the elongate member to return to an uncompressed state, thereby assisting the chamber of the heart to expand during diastole.

3. The method of claim 1, wherein providing the anchoring members comprises providing anchoring pads at the distal and the proximal end of the elongate member.

4. The method of claim 1, wherein the heart chamber is a left ventricle.

5. The method of claim 1, further comprising providing a force dispersion component fixedly secured to the distal end of the elongate member.

6. The method of claim 1, further comprising providing a force dispersion component fixedly secured to the proximal end of the elongate member.

7. The method of claim 1, further comprising providing a plurality of force dispersion components fixedly secured to the distal end and the proximal end of the elongate member.

9. A heart treatment device for improving the function of the heart, comprising:
a plurality of anchoring members;
an elongate member configured to be positioned transverse a chamber of the heart; where the elongate member has a substantially rigid distal end, a substantially rigid proximal end and a substantially elastic portion between the distal end and the proximal end; and
a release mechanism connected to the elongate member, the release mechanism being configured to releasably engage with each of a plurality of anchoring members having differing configurations to releasably attach the elongate member to each of the plurality of anchoring members one at a time.

10. The device of claim 9, wherein elasticity of the substantially elastic portion is configured to compress the elastic portion to retain energy during systole when subjected to contractile forces of the heart.

11. The device of claim 9, wherein elasticity of the substantially elastic portion is configured to cause the elongate member to exert expansive forces on the heart during diastole.

12. The device of claim 9, wherein each of the plurality of anchoring members defines a recess configured to receive the release mechanism.

13. The device of claim 12, wherein the recess is configured to receive the release mechanism when the release mechanism is in a first position.

14. The device of claim 9, further comprising a projection defining a portion of the recess, the projection being configured to prevent passage of the release mechanism through each of the plurality of anchoring members when the release mechanism is in the first position.

15. The device of claim 14, wherein the projection defines an opening configured to permit passage of the release mechanism therethrough when the release mechanism is in a second position.

16. The device of claim 9, wherein the release mechanism is in the form of a block.

17. The device of claim 9, wherein the release mechanism is moveable relative to the elongate member.

18. The device of claim 9, wherein the release mechanism is configured to articulate relative to the elongate member.

19. The device of claim 9, wherein the release mechanism is moveable between a first position wherein the release mechanism is releasable from each of the plurality of anchoring members and a second position wherein the release mechanism is engageable with each of the plurality of anchoring members.

20. The device of claim 9, further comprising at least one of the plurality of anchoring members having differing configurations.

21. The device of claim 9, further comprising an additional anchoring member for securing the elongate member relative to the heart, the additional anchoring member being configured to be attached to the elongate member.

22. The device of claim 21, wherein the at least one anchoring member is configured to be connected to the elongate member at the distal end of the elongate member and the additional anchoring member is configured to be attached to the elongate member at a second end of the elongate member.

23. The device of claim 21, wherein the at least one anchoring member is configured to be positioned on a posterior side of a mitral valve and wherein the additional anchoring member is configured to be positioned on an anterior side of the mitral valve.

24. The device of claim 21, wherein each of the at least one anchoring member and the additional anchoring member is configured to be positioned on an exterior surface of a wall of the heart.

25. The device of claim 21, wherein the at least one anchoring member comprises a first contact zone and a second contact zone, the first and second contact zones being configured to rest on an exterior surface of a wall of the heart.

26. The device of claim 21, wherein the at least one anchoring member further comprises a bridge connecting the first contact zone and the second contact zone.

27. The device of claim 21, wherein the first contact zone is configured to be positioned adjacent an annulus of a mitral valve and wherein the second contact zone is configured to be positioned approximately at a level of papillary muscles of the mitral valve.

28. The device of claim 21, wherein the first contact zone is configured to be positioned on a posterior side of the annulus.

29. The device of claim 22, wherein the bridge defines a recess configured to receive the release mechanism.

30. The device of claim 16, further comprising a covering on at least a portion of the at least one anchoring member.

31. The device of claim 26, wherein the covering is configured to facilitate tissue ingrowth.

32. The device of claim 9, wherein the plurality of differing anchoring members include anchoring members having differing sizes and/or shapes.

33. The device of claim 9, wherein the device is configured to improve valve function.

34. The device of claim 9, wherein the chamber is a left ventricle.

35. The device of claim 9, further comprising a force dispersion component fixedly secured to the distal end of the elongate member.

36. The device of claim 9, further comprising a force dispersion component fixedly secured to the proximal end of the elongate member.

37. The device of claim 9, further comprising providing a plurality of force dispersion components fixedly secured to the distal end and the proximal end of the elongate member.

38. The device of claim 37, wherein the force dispersion components are self expandable.

39. The device of claim 37, wherein the force dispersion components are similar to anchoring members.
